(19) United States
(12) Patent Application Publication
(10) Pub. No.: US 2021/0079470 A1
(43) Pub. Date: Mar. 18, 2021

(54) NONINVASIVE PRENATAL DIAGNOSIS OF SINGLE-GENE DISORDERS USING DROPLET DIGITAL PCR

(71) Applicants: Chan Zuckerberg Biohub, Inc., San Francisco, CA (US); The Board of Trustees of the Leland Standford Junior University, Stanford, CA (US)

(72) Inventors: Stephen Quake, Stanford, CA (US); Joan Camunas-Soler, Stanford, CA (US)

(21) Appl. No.: 16/626,187

(22) PCT Filed: Jul. 6, 2018
(86) PCT No.: PCT/US18/41150

§ 371 Date: Dec. 23, 2019

Related U.S. Application Data
(60) Provisional application No. 62/530,041, filed on Jul. 7, 2017.

(51) Int. Cl. C12Q 1/6883; G16B 5/20; C12Q 1/6851

(52) U.S. Cl. CPC C12Q 1/6883; C12Q 2600/156; C12Q 1/6851; G16B 5/20

(57) ABSTRACT

Methods for detection of single nucleotide mutations of autosomal recessive diseases as early as the first trimester of pregnancy are provided. This is of importance for metabolic disorders where early diagnosis can affect management of the disease and reduce complications and anxiety related to invasive testing.

### CROSS-REFERENCE TO RELATED APPLICATIONS

This application claims priority to U.S. Provisional Application No. 62/530,041, filed on Jul. 7, 2017, the disclosure of which is hereby incorporated by reference in its entirety for all purposes.

### BACKGROUND

The presence of circulating cell-free DNA (cfDNA) of fetal origin in maternal plasma has allowed the development of noninvasive tools to detect fetal genetic abnormalities from a maternal blood draw. Currently, noninvasive prenatal testing (NIPT) of common aneuploidies (e.g. Down's syndrome) is clinically available as a screening test that can be performed as early as week 10 of pregnancy without the complications related to invasive testing. More recently, NIPT has also become commercially available for some genomic microdeletions.

Prenatal diagnosis of pregnancies at risk of single gene disorders still requires the use of invasive techniques such as amniocentesis or chorionic villus sampling (CVS). These methods have a risk of miscarriage, can cause higher discomfort, and can only be applied during certain time windows of pregnancy.

The present disclosure provides methods and systems for noninvasive prenatal detection and/or diagnosis of inherited single gene disorders using droplet digital PCR (ddPCR) by analyzing circulating cell-free DNA (cfDNA) in maternal plasma.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

The present disclosure provides methods of diagnosing a single gene disorder in a fetus comprising: (a) quantifying total cell-free DNA (cfDNA) and a fetal fraction in a non-cellular fraction of a whole blood sample obtained from a pregnant subject, wherein the quantifying comprises an amplification-based multiple single nucleotide polymorphism (SNP) genotyping; and (b) quantifying a ratio of healthy and diseased alleles for a single gene disorder in the non-cellular fraction, wherein the quantifying comprises an amplification-based procedure.

In some embodiments, methods of diagnosing a single gene disorder in a fetus comprise: (a) quantifying total cell-free DNA (cfDNA) and a fetal fraction in a non-cellular fraction of a whole blood sample obtained from a pregnant subject, wherein the quantifying comprises an amplification-based multiple single nucleotide polymorphism (SNP) genotyping; (b) quantifying a ratio of healthy and diseased alleles for a single gene disorder in the non-cellular fraction, wherein the quantifying comprises an amplification-based procedure; and (c) applying a likelihood ratio classifier to the ratio of healthy and diseased alleles to diagnose a single gene disorder in a fetus of the pregnant subject.

In some embodiments, the methods of diagnosing a single gene disorder in a fetus comprise: (a) quantifying a fetal fraction in a non-cellular fraction of a whole blood sample obtained from a pregnant subject, wherein the quantifying comprises an amplification-based multiple single nucleotide polymorphism (SNP) genotyping; (b) determining an expected ratio of healthy and diseases alleles for a single gene disorder in the non-cellular fraction; (c) quantifying an actual ratio of healthy and diseased alleles of a single gene disorder in the non-cellular fraction, wherein the quantifying comprises an amplification procedure; and (d) comparing the expected ratio with the actual ratio to diagnose a single gene disorder in a fetus of the pregnant subject.

The present disclosure provides methods of quantifying a fetal fraction in a non-cellular fraction of a whole blood sample from a pregnant subject comprising: (a) performing amplification-based multiple single nucleotide polymorphism (SNP) genotyping and amplification-based chromosomal genotyping of cell-free DNA (cfDNA) in a non-cellular fraction of a whole blood sample from a pregnant subject; (b) quantifying a minor allele fraction (MAF) for each SNP in the SNP genotyping; and (c) determining the fetal fraction as a median of a distribution of SNPs that are homozygous for the pregnant subject and heterozygous for a fetus of the pregnant subject.

In some embodiments, methods of quantifying a fetal fraction in a non-cellular fraction of a whole blood sample from a pregnant subject comprise: (a) performing amplification-based multiple single nucleotide polymorphism (SNP) genotyping and amplification-based chromosomal genotyping of cell-free DNA (cfDNA) in a non-cellular fraction of a whole blood sample from a pregnant subject; (b) quantifying a minor allele fraction (MAF) for each SNP in the SNP genotyping; and (c) determining the fetal fraction as a median of a distribution of SNPs that are: (1) homozygous for the pregnant subject and heterozygous for a fetus of the pregnant subject; and/or (2) heterozygous for the pregnant subject and homozygous for a fetus of the pregnant subject.

Provided herein is a method of diagnosing a single gene disorder in a fetus comprising: (a) quantifying total cell-free DNA (cfDNA) and a fetal fraction in a non-cellular fraction of a whole blood sample obtained from a pregnant subject, wherein the quantifying comprises an amplification-based multiple single nucleotide polymorphism (SNP) genotyping; and (b) quantifying a ratio of healthy and diseased alleles for a single gene disorder in the non-cellular fraction, wherein the quantifying comprises an amplification-based procedure.

In some embodiments, the pregnant subject is in the first trimester of pregnancy, second trimester of pregnancy or third trimester of pregnancy. In some embodiments, the pregnant subject is in a first trimester of pregnancy. In some embodiments, the pregnant subject is at least about 9 weeks pregnant, at least about 10 weeks pregnant, at least about 11 weeks pregnant, at least about 12 weeks pregnant, at least about 13 weeks pregnant, at least about 14 weeks pregnant, or at least about 15 weeks pregnant. In some embodiments, the pregnant subject is at least about 9 weeks pregnant. In some embodiments, the pregnant subject is at least about 10 weeks pregnant.

In some embodiments, the amplification-based SNP genotyping comprises 2 or more SNPs, 3 or more SNPs, 4 or more SNPs, 5 or more SNPs, 6 or more SNPs, 7 or more SNPs, 8 or more SNPs, 9 or more SNPs, 10 or more SNPs, 11 or more SNPs, 12 or more SNPs, 13 or more SNPs, or 14 or more SNPs. In some embodiments, the amplification-based SNP genotyping comprises 2 or more SNPs. In some embodiments, the amplification-based SNP genotyping comprises 14 or more SNPs. In some embodiments, the amplification-based SNP genotyping comprises 47 SNPs. In some embodiments, at least one SNP comprises a SNP of Table 1.

In some embodiments, a fetal fraction of at least 1.0%, at least 1.5%, at least 2.0%, at least 2.5%, at least 3.0%, at least 3.5% or at least 4.0% is determined in step (a). In some embodiments, a fetal fraction of at least 2.0% is determined in step (a).

In some embodiments, the method described herein further comprises applying a likelihood ratio classifier to the ratio of healthy and diseased alleles to diagnose the single gene disorder in the fetus.

In some embodiments, the amplification-based SNP genotyping of step (a) comprises polymerase chain reaction (PCR), ligase chain reaction, transcription amplification, self-sustained sequence replication, or a combination thereof. In some embodiments, the amplification-based SNP genotyping of step (a) comprises droplet digital polymerase chain reaction (PCR).

In some embodiments, the amplification-based procedure of step (b) comprises polymerase chain reaction (PCR), ligase chain reaction, transcription amplification, self-sustained sequence replication, or a combination thereof. In some embodiments, the amplification-based procedure of step (b) comprises droplet digital polymerase chain reaction (PCR).

In some embodiments, the single gene disorder is an X-linked disorder, an autosomal recessive disorder, a compound heterozygous disorder, or a combination thereof. In some embodiments, the single gene disorder is an X-linked disorder. In some embodiments, the single gene disorder is an autosomal recessive disorder. In some embodiments, the single gene disorder is a compound heterozygous disorder. In some embodiments, the single gene disorder is selected from a group consisting of hemophilia A, hemophilia B, ornithine transcarbamylase deficiency (OTC), β-thalassemia, mevalonate kinase deficiency (MKD), muscle-type acetylcholine receptor (AChR) deficiency, cystic fibrosis, and GJB-2 related DFNB1 nonsyndromic hearing loss.

In some embodiments, the whole blood sample is debulked to obtain the non-cellular fraction. In some embodiments, steps (a) and (b) do not require genotyping of the pregnant subject.

Described herein is a method of diagnosing a single gene disorder in a fetus comprising: (a) quantifying total cell-free DNA (cfDNA) and a fetal fraction in a non-cellular fraction of a whole blood sample obtained from a pregnant subject, wherein the quantifying comprises an amplification-based multiple single nucleotide polymorphism (SNP) genotyping; (b) quantifying a ratio of healthy and diseased alleles for a single gene disorder in the non-cellular fraction, wherein the quantifying comprises an amplification-based procedure; and (c) applying a likelihood ratio classifier to the ratio of healthy and diseased alleles to diagnose a single gene disorder in a fetus of the pregnant subject.

In some embodiments, n the pregnant subject is in the first trimester of pregnancy, second trimester of pregnancy or third trimester of pregnancy. In some embodiments, the pregnant subject is in a first trimester of pregnancy. In some embodiments, the pregnant subject is at least about 9 weeks pregnant, at least about 10 weeks pregnant, at least about 11 weeks pregnant, at least about 12 weeks pregnant, at least about 13 weeks pregnant, at least about 14 weeks pregnant, or at least about 15 weeks pregnant. In some embodiments, the pregnant subject is at least about 9 weeks pregnant. In some embodiments, the pregnant subject is at least about 10 weeks pregnant.

In some embodiments, the amplification-based multiple SNP genotyping comprises 2 or more SNPs, 3 or more SNPs, 4 or more SNPs, 5 or more SNPs, 6 or more SNPs, 7 or more SNPs, 8 or more SNPs, 9 or more SNPs, 10 or more SNPs, 11 or more SNPs, 12 or more SNPs, 13 or more SNPs, or 14 or more SNPs. In some embodiments, the amplification-based multiple SNP genotyping comprises 2 or more SNPs. In some embodiments, the amplification-based multiple SNP genotyping comprises 14 or more SNPs. In some embodiments, the amplification-based multiple SNP genotyping comprises 47 SNPs. In some embodiments, at least one SNP comprises a SNP of Table 1.

In some embodiments, a fetal fraction of at least 1.0%, at least 1.5%, at least 2.0%, at least 2.5%, at least 3.0%, at least 3.5% or at least 4.0% is determined in step (a). In some embodiments, a fetal fraction of at least 2.0% is determined in step (a).

In some embodiments, the amplification-based multiple SNP genotyping of step (a) comprises polymerase chain reaction (PCR), ligase chain reaction, transcription amplification, self-sustained sequence replication, or a combination thereof. In some embodiments, the amplification-based multiple SNP genotyping of step (a) comprises droplet digital polymerase chain reaction (PCR).

In some embodiments, the amplification-based procedure of step (b) comprises polymerase chain reaction (PCR), ligase chain reaction, transcription amplification, self-sustained sequence replication, or a combination thereof. In some embodiments, the amplification-based procedure of step (b) comprises droplet digital polymerase chain reaction (PCR).

In some embodiments, the single gene disorder is an X-linked disorder, an autosomal recessive disorder, a compound heterozygous disorder, or a combination thereof. In some embodiments, the single gene disorder is an X-linked disorder. In some embodiments, the single gene disorder is an autosomal recessive disorder. In some embodiments, the single gene disorder is a compound heterozygous disorder. In some embodiments, the single gene disorder is selected from a group consisting of hemophilia A, hemophilia B, ornithine transcarbamylase deficiency (OTC), β-thalassemia, mevalonate kinase deficiency (MKD), muscle-type acetylcholine receptor (AChR) deficiency, cystic fibrosis, and GJB-2 related DFNB1 nonsyndromic hearing loss.

In some embodiments, the whole blood sample is debulked to obtain the non-cellular fraction. In some embodiments, steps (a)-(c) do not require genotyping of the pregnant subject.

Provided herein is a method of diagnosing a single gene disorder in a fetus comprising: (a) quantifying a fetal fraction in a non-cellular fraction of a whole blood sample obtained from a pregnant subject, wherein the quantifying comprises an amplification-based multiple single nucleotide polymorphism (SNP) genotyping; (b) determining an expected ratio of healthy and diseases alleles for a single gene disorder in the non-cellular fraction; (c) quantifying an actual ratio of healthy and diseased alleles of a single gene disorder in the non-cellular fraction, wherein the quantifying comprises an amplification procedure; and (d) comparing the expected ratio with the actual ratio to diagnose a single gene disorder in a fetus of the pregnant subject.

In some embodiments, the pregnant subject is in the first trimester of pregnancy, second trimester of pregnancy or third trimester of pregnancy. In some embodiments, the pregnant subject is in a first trimester of pregnancy. In some embodiments, the pregnant subject is at least about 9 weeks pregnant, at least about 10 weeks pregnant, at least about 11 weeks pregnant, at least about 12 weeks pregnant, at least about 13 weeks pregnant, at least about 14 weeks pregnant, or at least about 15 weeks pregnant. In some embodiments, the pregnant subject is at least about 9 weeks pregnant. In some embodiments, the pregnant subject is at least about 10 weeks pregnant.

In some embodiments, the amplification-based multiple SNP genotyping comprises 2 or more SNPs, 3 or more SNPs, 4 or more SNPs, 5 or more SNPs, 6 or more SNPs, 7 or more SNPs, 8 or more SNPs, 9 or more SNPs, 10 or more SNPs, 11 or more SNPs, 12 or more SNPs, 13 or more SNPs, or 14 or more SNPs. In some embodiments, the amplification-based multiple SNP genotyping comprises 2 or more SNPs. In some embodiments, the amplification-based multiple SNP genotyping comprises 14 or more SNPs. In some embodiments, the amplification-based multiple SNP genotyping comprises 47 SNPs. In some embodiments, at least one SNP comprises a SNP of Table 1.

In some embodiments, the amplification-based multiple SNP genotyping of step (a) comprises polymerase chain reaction (PCR), ligase chain reaction, transcription amplification, self-sustained sequence replication, or a combination thereof. In some embodiments, the amplification-based multiple SNP genotyping of step (a) comprises droplet digital polymerase chain reaction (PCR). In some embodiments, the amplification-based procedure of step (c) comprises polymerase chain reaction (PCR), ligase chain reaction, transcription amplification, self-sustained sequence replication, or a combination thereof. In some embodiments, wherein the amplification-based procedure of step (c) comprises droplet digital polymerase chain reaction (PCR).

In some embodiments, the single gene disorder is an X-linked disorder, an autosomal recessive disorder, a compound heterozygous disorder, or a combination thereof. In some embodiments, the single gene disorder is an X-linked disorder. In some embodiments, the single gene disorder is an autosomal recessive disorder. In some embodiments, the single gene disorder is a compound heterozygous disorder. In some embodiments, the single gene disorder is selected from a group consisting of hemophilia A, hemophilia B, ornithine transcarbamylase deficiency (OTC), β-thalassemia, mevalonate kinase deficiency (MKD), muscle-type acetylcholine receptor (AChR) deficiency, cystic fibrosis, and GJB-2 related DFNB1 nonsyndromic hearing loss.

In some embodiments, the whole blood sample is debulked to obtain the non-cellular fraction. In some embodiments, steps (a)-(d) do not require genotyping of the pregnant subject.

Described herein is a method of quantifying a fetal fraction in a non-cellular fraction of a whole blood sample from a pregnant subject comprising: (a) performing amplification-based multiple single nucleotide polymorphism (SNP) genotyping and amplification-based chromosomal genotyping of cell-free DNA (cfDNA) in a non-cellular fraction of a whole blood sample from a pregnant subject; (b) quantifying a minor allele fraction (MAF) for each SNP in the SNP genotyping; and (c) determining the fetal fraction as a median of a distribution of SNPs that are homozygous for the pregnant subject and heterozygous for a fetus of the pregnant subject.

In some embodiments, the pregnant subject is in the first trimester of pregnancy, second trimester of pregnancy or third trimester of pregnancy. In some embodiments, the pregnant subject is in a first trimester of pregnancy. In some embodiments, the pregnant subject is at least about 9 weeks pregnant, at least about 10 weeks pregnant, at least about 11 weeks pregnant, at least about 12 weeks pregnant, at least about 13 weeks pregnant, at least about 14 weeks pregnant, or at least about 15 weeks pregnant. In some embodiments, the pregnant subject is at least about 9 weeks pregnant. In some embodiments, wherein the pregnant subject is at least about 10 weeks pregnant.

In some embodiments, the amplification-based multiple SNP genotyping comprises 2 or more SNPs, 3 or more SNPs, 4 or more SNPs, 5 or more SNPs, 6 or more SNPs, 7 or more SNPs, 8 or more SNPs, 9 or more SNPs, 10 or more SNPs, 11 or more SNPs, 12 or more SNPs, 13 or more SNPs, or 14 or more SNPs. In some embodiments, the amplification-based multiple SNP genotyping comprises 2 or more SNPs. In some embodiments, the amplification-based multiple SNP genotyping comprises 14 or more SNPs. In some embodiments, the amplification-based multiple SNP genotyping comprises 47 SNPs. In some embodiments, at least one SNP comprises a SNP of Table 1.

In some embodiments, a fetal fraction of at least 1.0%, at least 1.5%, at least 2.0%, at least 2.5%, at least 3.0%, at least 3.5% or at least 4.0% is determined in step (c). In some embodiments, a fetal fraction of at least 2.0% is determined in step (c).

In some embodiments, the amplification-based multiple SNP genotyping of step (a) comprises polymerase chain reaction (PCR), ligase chain reaction, transcription amplification, self-sustained sequence replication, or a combination thereof. In some embodiments, the amplification-based multiple SNP genotyping of step (a) comprises droplet digital polymerase chain reaction (PCR). In some embodiments, the amplification-based procedure of step (a) comprises polymerase chain reaction (PCR), ligase chain reaction, transcription amplification, self-sustained sequence replication, or a combination thereof. In some embodiments, the amplification-based procedure of step (a) comprises droplet digital polymerase chain reaction (PCR).

In some embodiments, the whole blood sample is debulked to obtain the non-cellular fraction. In some embodiments, steps (a)-(c) do not require genotyping of the pregnant subject.

Provided herein is a method of quantifying a fetal fraction in a non-cellular fraction of a whole blood sample from a pregnant subject comprising: (a) performing amplification-based multiple single nucleotide polymorphism (SNP) genotyping and amplification-based chromosomal genotyping of cell-free DNA (cfDNA) in a non-cellular fraction of a whole blood sample from a pregnant subject; (b) quantifying a minor allele fraction (MAF) for each SNP in the SNP genotyping; and (c) determining the fetal fraction as a median of a distribution of SNPs that are: (1) homozygous for the pregnant subject and heterozygous for a fetus of the pregnant subject; and/or (2) heterozygous for the pregnant subject and homozygous for a fetus of the pregnant subject.

In some embodiments, the pregnant subject is in the first trimester of pregnancy, second trimester of pregnancy or third trimester of pregnancy. In some embodiments, the pregnant subject is in a first trimester of pregnancy. In some embodiments, the pregnant subject is at least about 9 weeks pregnant, at least about 10 weeks pregnant, at least about 11 weeks pregnant, at least about 12 weeks pregnant, at least about 13 weeks pregnant, at least about 14 weeks pregnant, or at least about 15 weeks pregnant. In some embodiments, the pregnant subject is at least about 9 weeks pregnant. In some embodiments, the pregnant subject is at least about 10 weeks pregnant.

In some embodiments, the amplification-based multiple SNP genotyping comprises 2 or more SNPs, 3 or more SNPs, 4 or more SNPs, 5 or more SNPs, 6 or more SNPs, 7 or more SNPs, 8 or more SNPs, 9 or more SNPs, 10 or more SNPs, 11 or more SNPs, 12 or more SNPs, 13 or more SNPs, or 14 or more SNPs. In some embodiments, the amplification-based multiple SNP genotyping comprises 2 or more SNPs. In some embodiments, the amplification-based multiple SNP genotyping comprises 14 or more SNPs. In some embodiments, the amplification-based multiple SNP genotyping comprises 47 SNPs. In some embodiments, at least one SNP comprises a SNP of Table 1.

In some embodiments, a fetal fraction of at least 1.0%, at least 1.5%, at least 2.0%, at least 2.5%, at least 3.0%, at least 3.5% or at least 4.0% is determined in step (c). In some embodiments, a fetal fraction of at least 2.0% is determined in step (c).

In some embodiments, the amplification-based multiple SNP genotyping of step (a) comprises polymerase chain reaction (PCR), ligase chain reaction, transcription amplification, self-sustained sequence replication, or a combination thereof. In some embodiments, the amplification-based multiple SNP genotyping of step (a) comprises droplet digital polymerase chain reaction (PCR). In some embodiments, the amplification-based procedure of step (a) comprises polymerase chain reaction (PCR), ligase chain reaction, transcription amplification, self-sustained sequence replication, or a combination thereof.

In some embodiments, the whole blood sample is debulked to obtain the non-cellular fraction. In some embodiments, steps (a)-(c) do not require genotyping of the pregnant subject.

### BRIEF DESCRIPTION OF THE DRAWINGS

The features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings or figures (also “FIG.” and “FIGs.” herein), of which:

FIG. 1 provides an exemplary protocol for noninvasive prenatal diagnostics of single-gene disorders, in accordance with some embodiments.

FIG. 2 provides an exemplary validation of diagnostic assays with synthetic spike in controls (g-blocks), in accordance with some embodiments.

FIG. 3 provides exemplary determination and quantification of cfDNA and fetal fractions, in accordance with some embodiments.

FIG. 4 provides exemplary diagnoses of fetuses at risk of maternally-inherited mutations, in accordance with some embodiments.

FIG. 5 provides exemplary diagnoses of fetuses at risk of combined paternal and maternal mutations for the same gene, in accordance with some embodiments.

FIG. 6 provides exemplary validation of diagnostic assays using fragmented genomic DNA (gDNA), in accordance with some embodiments.

FIG. 7 provides exemplary scatter plots showing spike in of synthetic DNA carrying mutation c.835C>T (OTC gene) in control gDNA, in accordance with some embodiments.

FIG. 8 provides exemplary fetal fraction determination using high-variability SNPs, in accordance with some embodiments.

FIG. 9 provides exemplary minor allele fraction (MAF) analyses for the fetal fraction assay at different time-points of a pregnancy at risk of mevalonate kinase (MVK) deficiency, in accordance with some embodiments.

FIG. 10 provides an exemplary analysis of a pregnancy at risk of ornithine transcarbamylase (OTC) deficiency (c.835C>T) due to maternal gamete mosaicism, in accordance with some embodiments.

FIG. 11 provides an exemplary analysis of a pregnancy at risk of OTC deficiency (c.67C>T), in accordance with some embodiments.

FIG. 12 provides an exemplary analysis of a pregnancy at risk of GJB2-related DFNB1 nonsyndromic hearing loss due to a heterozygous compound mutation, in accordance with some embodiments.

FIG. 13 provides an exemplary scheme of fractions expected in maternal plasma in an X-linked disease, in accordance with some embodiments.

FIG. 14 provides an exemplary scheme of fractions expected in maternal plasma in an autosomal recessive disease, in accordance with some embodiments.

### DETAILED DESCRIPTION

The presence of circulating cell-free DNA (cfDNA) of fetal and placental origin in maternal plasma has allowed the development of noninvasive tools to detect fetal genetic abnormalities from a maternal blood draw. Currently, noninvasive prenatal testing (NIPT) of common aneuploidies (e.g. Down syndrome) is clinically available as a screening test that can be performed as early as week 10 of pregnancy with false positive rates below 0.2% and without the complications related to invasive testing. More recently, NIPT has also become commercially available for some genomic microdeletions (e.g. DiGeorge syndrome, Cri-du-chat syndrome).

However, prenatal diagnosis of pregnancies at risk of single gene disorders still requires the use of invasive techniques such as amniocentesis or chorionic villus sampling (CVS). These methods have a risk of miscarriage, can cause higher discomfort, and can only be applied during certain time windows of pregnancy. Although commercial development of screening tests for single gene disorders is difficult due to the low prevalence of each given mutation in the general population (hampering positive predictive values), the development of accurate NIPT could replace invasive testing and become a diagnostic test for parents who are carriers of a mutation, who are at high risk of having an affected pregnancy.

The development of noninvasive tools for these disorders is important as it allows patients and doctors to make informed decisions in pregnancies at risk of severe conditions while reducing anxiety related to invasive or postnatal testing. In addition early treatment is sometimes available for conditions that might otherwise cause irreversible damage to the fetus such as metabolic disorders or congenital malformations (e.g. dietary treatment or neonatal surgery respectively). Finally, prenatal diagnostics might also prove useful to develop protocols for cord blood collection in views of potential cures of inherited single-gene disorders by using gene-editing techniques on hematopoietic stem cells.

Detection of single-gene disorders is straight-forward for paternally-inherited mutations or common de novo mutations, where the presence of a mutated allele in maternal plasma can be directly attributed to an affected fetus and not to background cfDNA of maternal origin. However, most common single-gene disorders are autosomal-recessive due to their deleterious nature, and therefore one must carefully quantitate the ratio of mutant to wild type alleles in order to genotype the fetus. This problem has been solved in principle by applying the counting principle to high depth whole exome sequence or to full haplotypes, but this approach requires the use of sequencing and is more costly than digital PCR. Proof-of-concept studies with digital PCR have been conducted for a number of autosomal recessive and X-linked disorders, but a general method to perform noninvasive diagnosis of these conditions is not yet available. Previous digital PCR studies have been limited in that they have not had large enough SNP panels to measure the fetal fraction in the general population, or have not had enough SNP measurements to estimate the error in measurement of fetal fraction.

In the present disclosure these challenges have been addressed by developing a droplet digital PCR (ddPCR) protocol to diagnose autosomal and X-linked single gene disorders. This protocol may be applied directly to the maternal cell free DNA sample and may not require a separate maternal genotyping step. An accurate quantification of the fetal fraction can be achieved by targeting a panel of 47 high-variability SNPs, and the final measurement error in determining fetal genotype may be composed of roughly equal contributions from the error in fetal fraction and the Poisson error due to counting statistics. This method may enable the diagnosis of recessive single gene disorders, both when they are due to a mutation shared by both progenitors or to heterozygous compound mutations (when father and mother carry a different mutation affecting the same gene). In some cases, unambiguous results may be calculated for samples with a fetal fraction less than 3.6%.

In the present disclosure, a direct ddPCR approach to test pregnancies at risk of X-linked and autosomal recessive single-gene disorders both for single mutations and compound heterozygous mutations has been presented. In some embodiments, the protocol does not require extensive sample preparation or computational resources. In some embodiments, noninvasive prenatal diagnosis can be performed in a clinical laboratory setting in ˜1 day from sample collection. This is particularly relevant for single-gene disorders where samples typically come in sparsely and are rarely at-risk for the same mutation. This approach may be validated by correctly diagnosing pregnancies at risk of some of the most common point mutations such as ΔF508 (accounting for >70% of cystic fibrosis cases in Europe), as well as rare metabolic and neuromuscular disorders that had not yet been addressed using non-invasive techniques (e.g. OTC, MKD or AchR deficiency). Early diagnosis of these metabolic disorders may improve management of the disease, especially in cases where early onset might lead to the accumulation of metabolites that cause irreversible organ damage and failure.

The current disclosure provides a method to measure the fetal fraction and total amount of cfDNA in plasma samples using a multiplexed SNP panel for ddPCR. This approach may be used to establish confidence intervals in NIPT of autosomal recessive or X-linked diseases. NIPT of these conditions relies on comparing the ratio of mutated and healthy alleles in maternal plasma to the ratios expected for a healthy or affected fetus as determined from the sample fetal fraction. Overall, the use of a SNP panel instead of a single marker to measure fetal fraction may be used to (i) reduce false positive and negative rates, (ii) reduce sample dropout due to a lack of indicative markers, and (iii) simplify the workflow as an initial maternal genotyping step may not be needed.

In some embodiments, a protocol for noninvasive prenatal diagnosis of inherited single gene disorders using droplet digital PCR (ddPCR) from circulating cell-free DNA (cfDNA) in maternal plasma may be used. First, the amount of cfDNA and fetal fraction may be determined using a panel of Taqman assays targeting high-variability SNPs. Second, the ratio of healthy and diseased alleles in maternal plasma may be quantified using Taqman assays targeting the mutations carried by the parents.

The development of noninvasive tools for single gene disorders discussed in the present disclosure may be used as a screening test or as a diagnostic tool for pregnancies where the progenitors are carriers of known mutations. The assays presented in this disclosure may be used in combination with carrier screening assays.

In some embodiments, a panel of diagnostic assays targeting the most common mutations involved in single gene disorders could be used as a noninvasive prenatal screening test for the general population (i.e. not known to be carriers of a mutation involved in a single gene disorder). In some cases, prenatal diagnosis of single gene disorders may be used by patients and doctors to make informed decisions in pregnancies at risk of severe conditions while reducing anxiety related to invasive or postnatal testing. In addition, the protocols discussed herein may be used to provide early treatments for single gene diseases that might otherwise cause irreversible damage to the fetus such as metabolic disorders or congenital malformations (e.g. dietary treatment or neonatal surgery respectively).

In some embodiments, the methods could be applied to develop protocols for cord blood collection in views of potential cures of inherited single-gene disorders by using gene-editing techniques (e.g. CRISPR) on hematopoietic stem cells. For instance, an application of this invention could be the development of a screening test to decide whether cord blood should be collected and stored upon delivery in a pregnancy at risk of a single gene disorder.

The methods discussed, may be used whenever one needs to establish confidence intervals in NIPT of autosomal recessive or X-linked diseases. Alternatively, they could be used for clinical applications to detect exogenous DNA in human biofluids or to monitor organ transplant rejection (by targeting the proposed set of SNPs used in the fetal fraction panel).

The methods described here may be used to validate diagnostic assays without the need of genomic DNA of a carrier of the mutation. In some embodiments, the methods can be applied to validation schemes for other mutations not related to prenatal diagnosis (e.g. screening for cancer mutations).

In some embodiments, the multiplexing of the diagnostic assay may be performed to detect several mutations at risk in a single experiment. This may be performed either using a preamplification scheme as that shown for the SNP panel, or by using different concentrations of primers/probes for each mutation to multiplex each individual ddPCR experiment.

The method developed here may be used to unambiguously test inheritance of single gene disorders using a maternal blood draw. The methods presented provide a direct scheme to diagnose inheritance of autosomal recessive and X-linked mutations in a noninvasive way using ddPCR.

The methods of the current disclosure provide screening tests for the inheritance of paternal mutations by: (1) developing a method for an accurate quantification of the fetal fraction and total amount of circulating cfDNA in maternal blood using a panel of genotyping assays and gene markers; (2) developing a method to validate ddPCR diagnostic assays for target mutations without the need of a genomic DNA sample from a carrier of the mutation; and (3) designing a method that allows to process samples regardless of being at risk of inheriting a mutation shared by both progenitors or at risk of inheriting different mutations from the father and mother. In some cases, the present disclosure presents a method of optimizing the split of sample used in each diagnostic test (paternal and maternal mutation). The split in sample may be used in cases of low abundance of fetal DNA in blood draws and where high statistics may be required to detect inheritance of a mutation carried by the mother.

The present disclosure provides methods of diagnosing a single gene disorder in a fetus comprising: (a) quantifying total cell-free DNA (cfDNA) and a fetal fraction in a non-cellular fraction of a whole blood sample obtained from a pregnant subject, wherein the quantifying comprises an amplification-based multiple single nucleotide polymorphism (SNP) genotyping; and (b) quantifying a ratio of healthy and diseased alleles for a single gene disorder in the non-cellular fraction, wherein the quantifying comprises an amplification-based procedure.

In some embodiments, methods of diagnosing a single gene disorder in a fetus comprise: (a) quantifying total cell-free DNA (cfDNA) and a fetal fraction in a non-cellular fraction of a whole blood sample obtained from a pregnant subject, wherein the quantifying comprises an amplification-based multiple single nucleotide polymorphism (SNP) genotyping; (b) quantifying a ratio of healthy and diseased alleles for a single gene disorder in the non-cellular fraction, wherein the quantifying comprises an amplification-based procedure; and (c) applying a likelihood ratio classifier to the ratio of healthy and diseased alleles to diagnose a single gene disorder in a fetus of the pregnant subject.

In some embodiments, the methods of diagnosing a single gene disorder in a fetus comprise: (a) quantifying a fetal fraction in a non-cellular fraction of a whole blood sample obtained from a pregnant subject, wherein the quantifying comprises an amplification-based multiple single nucleotide polymorphism (SNP) genotyping; (b) determining an expected ratio of healthy and diseases alleles for a single gene disorder in the non-cellular fraction; (c) quantifying an actual ratio of healthy and diseased alleles of a single gene disorder in the non-cellular fraction, wherein the quantifying comprises an amplification procedure; and (d) comparing the expected ratio with the actual ratio to diagnose a single gene disorder in a fetus of the pregnant subject.

The present disclosure provides methods of quantifying a fetal fraction in a non-cellular fraction of a whole blood sample from a pregnant subject comprising: (a) performing amplification-based multiple single nucleotide polymorphism (SNP) genotyping and amplification-based chromosomal genotyping of cell-free DNA (cfDNA) in a non-cellular fraction of a whole blood sample from a pregnant subject; (b) quantifying a minor allele fraction (MAF) for each SNP in the SNP genotyping; and (c) determining the fetal fraction as a median of a distribution of SNPs that are homozygous for the pregnant subject and heterozygous for a fetus of the pregnant subject.

In some embodiments, methods of quantifying a fetal fraction in a non-cellular fraction of a whole blood sample from a pregnant subject comprise: (a) performing amplification-based multiple single nucleotide polymorphism (SNP) genotyping and amplification-based chromosomal genotyping of cell-free DNA (cfDNA) in a non-cellular fraction of a whole blood sample from a pregnant subject; (b) quantifying a minor allele fraction (MAF) for each SNP in the SNP genotyping; and (c) determining the fetal fraction as a median of a distribution of SNPs that are: (1) homozygous for the pregnant subject and heterozygous for a fetus of the pregnant subject; and/or (2) heterozygous for the pregnant subject and homozygous for a fetus of the pregnant subject.

### EXAMPLES

### Example 1: Sample Collection and cfDNA Extraction

A total of 10 blood samples were collected from pregnancies at risk of a single-gene disorder. Samples were collected in cfDNA Streck tubes (3 tubes, approximately 30 mL). Blood was centrifuged at 1,600 g for 10 minutes, and the supernatant was centrifuged for an additional 10 minutes at 16000 g to remove cellular debris. Plasma samples were aliquoted in 2 ml tubes and stored at −80° C. until further processing (cfDNA extraction). Maternal genomic DNA was extracted from the remaining cellular fraction using the Qiagen Blood Mini kit (200 μl aliquots), and stored for assay validation. Extraction of cfDNA from stored plasma samples was done using the Qiagen Circulating Nucleic Acid kit using the protocol recommended by the manufacturer with the following modifications: an initial centrifugation of plasma for 3 minutes at 14,000 rpm to remove cryoprecipitates was performed, the lysis step was extended to 1 h (as recommended for Streck tubes), and no carrier RNA was added. Plasma was processed in batches of 5 ml per Qiagen column and eluted in 50 μl TE buffer.

### Example 2: Quantification of cfDNA in Plasma and Fetal Fraction

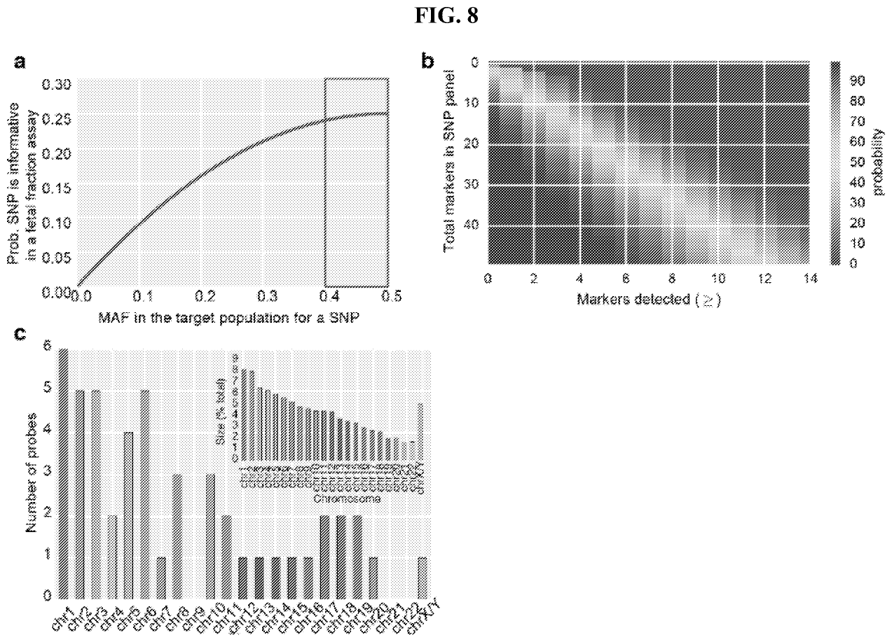

From the extracted cfDNA (˜150 μl in total) 8.5 μl (˜850 μl plasma) was used for a preamplification reaction targeting highly variable SNPs that was used to determine the fetal fraction of each sample. A total of 47 biallelic SNPs that show a high minor allele fraction (MAF>0.4) were selected for all five superpopulations of the 1000 genomes project (EAS, EUR, AFR, AMR, SAS) and that are not found in regions of structural variation or highly repetitive regions (filtered using UCSC RepeatMasker and the Database of Genomic Variants). Commercially available SNP Genotyping assays (ThermoFisher) were purchased for the selected SNPs (amplicon size <80 bp), as well as separate primers targeting each SNP region, as shown in Table 1. An additional SNP Taqman assay targeting the ZFX and ZFY genes in chromosomes X/Y was also included in the assay. The size of the SNP panel, threshold MAF, and chromosomal distribution of assays was designed to maximize the probability of making an accurate determination of the fetal fraction across a broad target population, as shown in FIG. 8.

TABLE 1
List of SNPs used for fetal fraction
quantification.

| | | ThermoFisher | |
|---|---|---|---|
| Name | dbSNP | Assay | Location |
| SNP1 | rs7549293 | C_9114654_10 | ch. 1: 205343152 |
| SNP2 | rs13218440 | C_9371416_10 | ch. 6: 12059721 |
| SNP3 | rs12423234 | C_488643_10 | ch. 12: 4821194 |
| SNP4 | rs1736442 | C_3285337_1_ | ch .18: 57558545 |
| SNP5 | rs1498553 | C_1452175_10 | ch. 11: 5687798 |
| SNP7 | rs1410059 | C_7538108_10 | ch. 10: 95412838 |
| SNP9 | rs2304102 | C_8582892_1_ | ch. 19: 32976451 |
| SNP10 | rs2256111 | C_12083303_10 | ch. 11: 117864047 |
| SNP12 | rs7325978 | C_29381390_10 | ch. 13: 73062760 |
| SNP13 | rs12148532 | C_31740865_10 | ch. 15: 73929859 |
| SNP14 | rs249290 | C_1724866_10 | ch. 16: 9477431 |
| SNP16 | rs1544724 | C_8727861_10 | ch. 17: 7621777 |
| SNP17 | rs3760269 | C_27475947_10 | ch. 17: 66289041 |
| SNP18 | rs7233004 | C_1527844_10 | ch. 18: 53516802 |
| SNP19 | rs4801945 | C_314514_10 | ch. 19: 39407014 |
| SNP20 | rs565522 | C_3106336_10 | ch. 1: 112261533 |
| SNP22 | rs2737654 | C_15837816_10 | ch. 1: 200046444 |
| SNP23 | rs2576241 | C_96592021_10 | ch. 1: 217100192 |
| SNP24 | rs1914748 | C_11509308_10 | ch. 2: 106035580 |
| SNP25 | rs12694624 | C_32049532_10 | ch. 2: 224837600 |
| SNP26 | rs6781236 | C_29259075_10 | ch. 3: 9163206 |
| SNP27 | rs7653090 | C_26033960_10 | ch. 3: 72554890 |
| SNP28 | rs357485 | C_1307096_10 | ch. 3: 153887666 |
| SNP30 | rs4975819 | C_365652_10 | ch. 5: 2103617 |
| SNP31 | rs6899022 | C_76234724_20 | ch. 5: 10506997 |
| SNP32 | rs6924733 | C_1661055_10 | ch. 6: 15385281 |
| SNP33 | rs2535290 | C_9436700_10 | ch. 6: 31063132 |
| SNP34 | rs172275 | C_1024320_10 | ch. 6: 32961621 |
| SNP35 | rs4644087 | C_27981057_10 | ch. 6: 127481154 |
| SNP36 | rs9792284 | C_26926661_20 | ch. 8: 3495692 |
| SNP37 | rs7827391 | C_9870422_10 | ch. 8: 32542912 |
| SNP38 | rs2319150 | C_8468497_10 | ch. 8: 96775900 |
| SNP39 | rs1160680 | C_9469291_10 | ch. 1: 19080506 |
| SNP42 | rs1399629 | C_1533279_20 | ch. 2: 240257958 |
| SNP44 | rs2276702 | C_15882282_10 | ch. 2: 1426621 |
| SNP46 | rs9290003 | C_9889051_10 | ch. 3: 99906993 |
| SNP47 | rs6802328 | C_402927_10 | ch. 3: 186088878 |
| SNP48 | rs17017347 | C_33246860_10 | ch. 4: 91573596 |
| SNP49 | rs10027026 | C_11856187_10 | ch. 4: 190371233 |
| SNP50 | rs1185246 | C_9934576_10 | ch. 5: 68715310 |
| SNP51 | rs6877199 | C_31986570_10 | ch. 5: 151274117 |
| SNP52 | rs12690832 | C_7641241_10 | ch. 7: 43173610 |
| SNP55 | rs10821808 | C_31345071_10 | ch. 10: 62390646 |
| SNP56 | rs2370764 | C_31230_10 | ch. 10: 32685008 |
| SNP57 | rs3742560 | C_9866252_1_ | ch. 14: 55106083 |
| SNP58 | rs271981 | C_2959256_10 | ch. 20: 58002599 |
| SNP59 | rs701232 | C_2469291 | ch. 1: 233655723 |
| X/Y | Forward | 5-CAAGTGCTGGA | |
| assay | | CTCAGATGTAACT | |
| | | GT-3 | |
| | Reverse | 5-TGAAGTAATGT | |
| | | CAGAAGCTAAAAC | |
| | | ATCA-3 | |
| | Probe X | 5-(FAM)TCTTTA | |
| | | CCACACTGCAC | |
| | | (MGBNFQ)-3 | |
| | Probe Y | 5-(VIC)TCTTTA | |
| | | GCACATTGCA | |
| | | (MGBNFQ)-3 | |

The preamplification reaction was performed using the Taqman PreAmp Master Mix (Applied Biosystems, Ref. 4391128) with the pooled 48 primer pairs and the recommended conditions by the manufacturer (reaction volume 50 μl, final primer concentration 45 nM each, 11 preamplification cycles). The preamplified DNA was diluted 5× with TE buffer and stored for ddPCR quantification.

Quantification of the fetal fraction and total amount of cfDNA were performed using ddPCR and standard conditions (reaction volume 20 μl, final primer (probe) concentration: 900 nM (200 nM), thermal cycling: [10′ 95° C.; 40×[30″ 94° C.; 1′ 60° C.]; 10′ 98° C.], ramp rate: 2° C./sec). 1 μl of the preamplified DNA for each SNP Taqman assay reaction and 1 μl of the original cfDNA for each quantification assay (Tables 1 and 2) were used. For the quantification assays 2 multiplex assays targeting chromosomes 1, 5, 10 and 14 (Table 2) were used. Fetal fraction and quantification ddPCR assays were run in parallel in a single plate.

TABLE 2
Assays used for cfDNA quantification.

| | Gene | | | Fluoro- | |
|---|---|---|---|---|---|
| Name | Target | Location | Size | phore | Reference |
| Assay | EIF2C1 | 1: 36359312- | 69 | FAM | Thermofisher/ |
| Q1 | | 36359434 | | | dHsaCP2500316 |
| | Rnase P | 14: 20811565 | 88 | VIC | BioRad/4403326 |
| Assay | RPP30 | 10: 92660373- | 67 | FAM | Thermofisher/ |
| Q2 | | 92660495 | | | dHsaCP2500313 |
| | TERT | 5: 5p15.33 | 87 | VIC | BioRad/4403316 |

The amount of cfDNA per ml of plasma (in genomic equivalents) is determined as the mean of the four quantification assays. For the SNP assays, Poisson corrected counts are determined as N_{FAM/VIC}=Nₜₒₜₐₗ ln[1−Nₚₒₛᵢₜᵢᵥₑ/Nₜₒₜₐₗ] (Equation 1), where Nₜₒₜₐₗ is the total number of droplets and Nₚₒₛᵢₜᵢᵥₑ the number of positive droplets for each channel (FAM or VIC). For each SNP assay the minor allele fraction is extracted as MAF=min(N_{FAM}, N_{VIC})/(N_{FAM}+N_{VIC}). The fetal fraction (ε) is determined from the median of all SNPs where the fetus is heterozygous and the mother homozygous (typically in the range 0.5%<MAF<20%) using ε=2MAF. This typically represents samples with a fetal fraction in the range of at least about 1% to at most about 40%. The assays for mutations at-risk can be performed for fetal fraction below 1% using SNPs that show a MAF<0.5% to measure the fetal fraction. Errors are determined as the standard deviation (SD) and compared to the Poisson noise expected from the DNA input used in the preamplification reaction (δε_{Poisson}=\sqrt{2ε/[input DNA in preamp]}).

### Example 3: Design and Validation of Assays for Mutations at-Risk

For each sample, the fetal fraction and total amount of cfDNA was initially determined as detailed above. From these values, the optimal split of sample between the paternal and maternal mutation was determined, as well as the probability of obtaining an unambiguous result. Assays to detect inheritance of the mutations were designed and validated as described below.

Assay Design

Primers and probes for each target mutation using Primer3 with parameters similar to those recommended for ddPCR (amplicon size ≤90 bp) were designed. Melting temperature for MGB probes was determined using PrimerExpress (ThermoFisher). Typically, 3 Taqman assays per mutation were designed and the best one was selected using the validation schemes described below. Sequences of primers and probes targeting the mutations studied in this work are found in Table 3, together with their amplicon size and optimal temperature for ddPCR (as determined in the validation assays).

TABLE 3
Sequences of primers and probes for the disease mutations tested.

| | | | Length |
|---|---|---|---|
| Disease | Primers | | Optim. |
| Gene (Mutation) | Probes | Sequence | Temp. |
| Hemophilia A | Forward | 5′-ACCACTCCTGAAGTGCACTC-3′ | 63 bp |
| F8 (c.1042G > A) | Reverse | 5′-GCGATGGTTCCTCACAAGAAA-3′ | 54.8° C. |
| | FAM-MGB | 5′-ATTCCTCAAAGGTCACA-3′ | |
| | VIC-MGB | 5′-ATTCCTCGAAGGTCACAC-3′ | |
| Hemophilia B | Forward | 5′-CGTGCCAATTCAATTTCTTAACC-3′ | 69 bp |
| F9 (c.278 − 1G > C) | Reverse | 5′-CATTTAAACATGGATTGGACTCACA-3′ | 56.8° C. |
| | FAM-MGB | 5′-TCTCAAACATGGAGATC-3′ | |
| | VIC-MGB | 5′-TCTCAAAGATGGAGATC-3′ | |
| β-thalassemia | Forward | 5′-TGGATGAAGTTGGTGGTGA-3′ | 69 bp |
| HBB (c.92 + 5G > C) | Reverse | 5′-TGGTCTCCTTAAACCTGTCT-3′ | 56.1° C. |
| rs33915217 | FAM-MGB | 5′-CAGGITGCTATCAAG-3′ | |
| | VIC-MGB | 5′-CAGGTTGGTATCAAG-3′ | |
| Mevalonate kinase | Forward | 5′-TCTCCATCCACTCAGCCACCT-3′ | 79 bp |
| deficiency | Reverse | 5′-AGTGTCGTGGGCTCCTCTCA-3′ | 56.1° C. |
| MKD (c.1162C > T) | FAM-MGB | 5′-CTGGACAGCTGAGTC-3′ | |
| | VIC-MGB | 5′-TGGACAGCCGAGTC-3′ | |
| Muscle-type acetyl- | Forward | 5′-CCTGCCATCTTCCGTTCC-3′ | 87 bp |
| choline receptor | Reverse | 5′-GCCTCACTGGAAGATAAGGG-3′ | 54.8° C. |
| CHRNG (c459dupA) | FAM-MGB | 5′-TCTATCTCAGTCAACC-3′ | |
| rs774279192 | VIC-MGB | 5′-TCTATCTCAGTCACCTAC-3′ | |
| Muscle-type acetyl- | Forward | 5′-GCAAGCCCCTCTTCTACGTC-3′ | 65 bp |
| choline receptor | Reverse | 5′-GATGGCGACAGAGGAGATGAG-3′ | 56.1° C. |
| CHRNG (c753_754delCT) | FAM-MGB | 5′-CATCGCCCCGTGTG-3′ | |
| rs767503038 | VIC-MGB | 5′-TCGCCCCCTGTGTG-3′ | |
| Cystic fibrosis | Forward | 5′-TGCCTGGCACCATTAAAGAA-3′ | 89 bp |
| CFTR (delF508) | Reverse | 5′-GCATGCTTTGATGACGCTTC-3′ | 56.1° C. |
| rs77010898 | FAM-MGB | 5′-ATATCATTGGTGTTTCC-3′ | |
| | VIC-MGB | 5′-ATATCATCTTTGGTGTTTC-3′ | |
| Cystic fibrosis | Forward | 5′-TGTGTCTTGGGATTCAATAACTTTG-3′ | 88 bp |
| CFTR (W1282X) | Reverse | 5′-TTTTTCTGGCTAAGTCCTTTTGCT-3′ | 60.2° C. |
| rs77010898 | FAM-MGB | 5′-AACAGTGAAGGAAAGC-3′ | |
| | VIC-MGB | 5′-ACAGTGGAGGAAAGC-3′ | |
| Ornithine transcarb- | Forward | 5′-GCATGGAGGCAATGTATTAATTACAG-3′ | 121 bp |
| amylase deficiency | Reverse | 5′-GGCATCAATTTGTACCTTCATTGT-3′ | 56.1° C. |
| OTC (c.835C > T) | FAM-MGB | 5′-AAAAAGCGGCTCTAG-3′ | |
| rs72558455 | VIC-MGB | 5′-AAAAAGCGGCTCCAG-3′ | |
| Ornithine transcarb- | Forward | 5′-TCCTGTTAAACAATGCAGCT-3′ | 82 bp |
| amylase deficiency | Reverse | 5′-CCCAAGTCTCTGACCATCAC-3′ | 56.1° C. |
| OTC (c.67C > T) | FAM-MGB | 5′-CCGAAAATTTCAAACCA-3′ | |
| rs72552300 | VIC-MGB | 5′-CCGAAAATTTCGAACCA-3′ | |
| DFNB1 non-syndromic | Forward | 5′-TGAACAAACACTCCACCAGC-3′ | 81 bp |
| hearing loss | Reverse | 5′-CAGCCACAACGAGGATCATA-3′ | 58° C. |
| GJB2 (c.71G > A) | FAM-MGB | 5′-CGGTGAGCTAGATCT-3′ | |
| rs104894396 | VIC-MGB | 5′-TGAGCCAGATCTT-3′ | |
| DFNB1 non-syndromic | Forward | 5′-CAACGCCGAGACCCCC-3′ | 94 bp |
| hearing loss | Reverse | 5′-GTTCCTGGCCGGGCAG-3′ | 62.1° C. |
| GJB2 (c.-23 + 1G > A) | FAM-MGB | 5′-ACGCAGATGAGCC-3′ | |
| rs80338940 | VIC-MGB | 5′-ACGCAGGTGAGCC-3′ | |

Assay Validation Using Carrier Genomic DNA

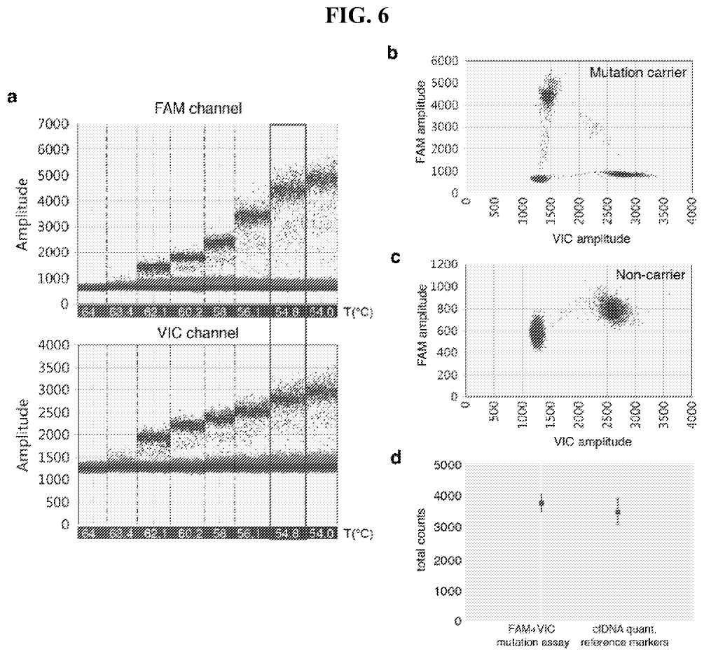

Validation of the assays was performed using genomic DNA that is heterozygous for the target mutation (one affected allele and one healthy allele). This approach could be used for maternally-inherited mutations (using gDNA from maternal blood cells) or when cell-line DNA was available from a biorepository (e.g. Coriell). Extracted gDNA from the carrier was fragmented to an average size of −150 bp using a Covaris S2 instrument and normalized to −15 ng/μ1 (−4000 genomic equivalents/μ1) as measured in a Qubit. A non-carrier male and female control were processed in the same way. The Taqman assays using a temperature gradient in ddPCR were validated (FIG. 6a,b). The assay and temperature giving the best separation for FAM and VIC channels and having a shorter amplicon size was typically selected. Standard quantification assays for each sample were ran in parallel to discard the presence of copy variants or pseudogenes in the target region (FIG. 6c).

Assay Validation Using Synthetic Spike in

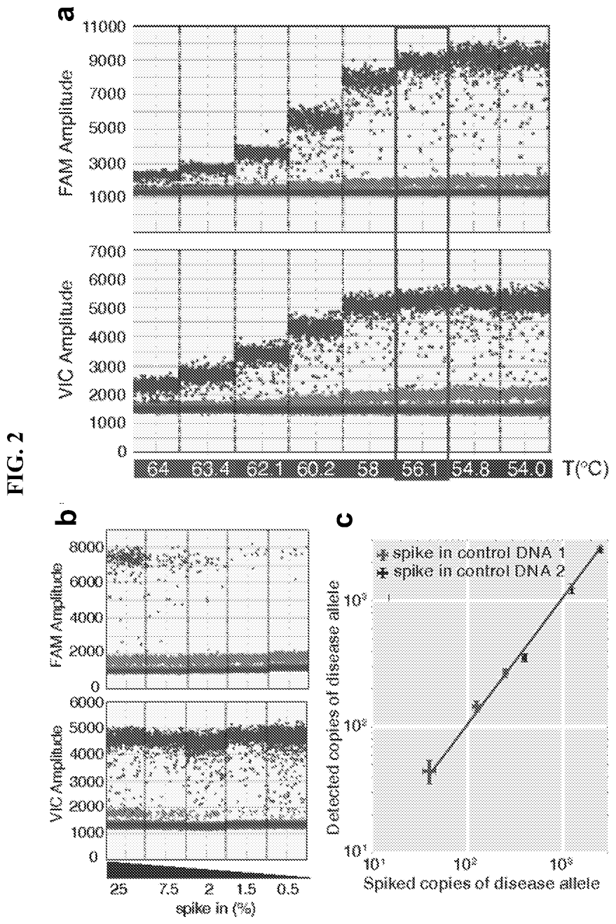

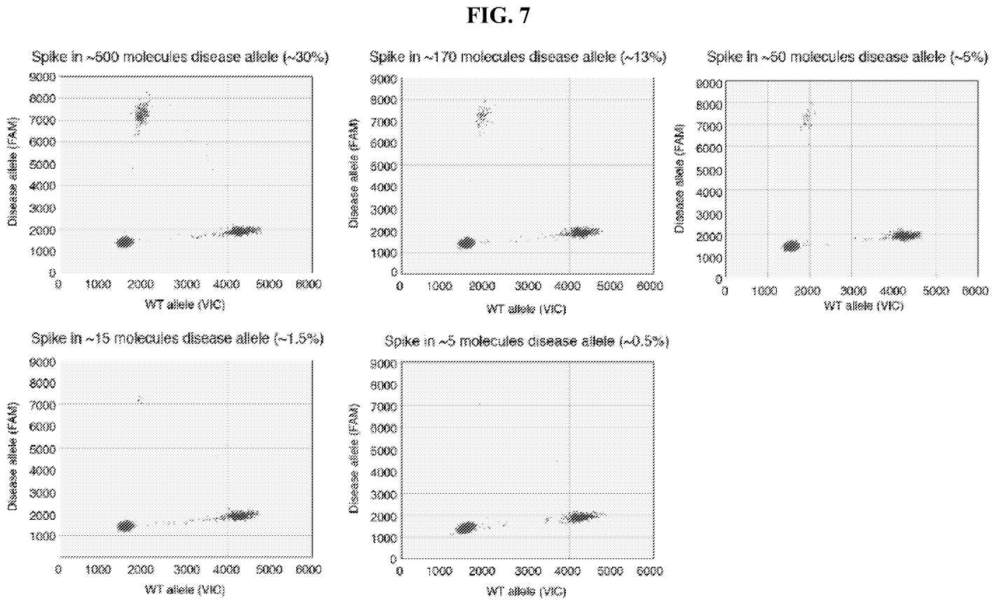

An alternative validation assay using synthetic spike in DNA was also developed for samples for which there was no gDNA from a carrier of the mutation. For that, two synthetic DNA fragments (gBlock, IDT) containing the target region of the assay were purchased: one containing the mutated allele and the other the healthy allele. The fragments were quantified and diluted to 5000 genomic equivalents/μ1 and mixed to a 1:1 ratio. The Taqman assays against this mixture using a temperature gradient in ddPCR was then validated (FIG. 2a). The best assay and temperature was picked and performed further validation by spiking in different amounts of the synthetic mutated-allele fragment into non-carrier genomic DNA controls (FIGS. 2b,c and 7). FIG. 7 illustrates the spike in of synthetic DNA carrying mutation c.835C>T (OTC gene) in control gDNA. Scatter plot of FAM/VIC fluorescence in ddPCR experiments where varying amounts of synthetic DNA carrying a mutated allele is spiked into a constant background of fragmented gDNA of a healthy female donor. These experiments are used to perform a validation plot as the one shown in FIG. 2c.

To test the paternal mutation the amount of cfDNA expected to provide ˜40 counts for a carrier fetus was used. This sets the result 6 standard deviations away from the non-carrier case. The remaining sample was used to quantify the imbalance on the maternal mutation. The ddPCR measurements were run using standard conditions and optimal temperatures determined in the validation assays. For each assay the total number of counts for each allele was determined using Equation 1. The affected or unaffected status of the fetus was determined using a likelihood ratio classifier with a low threshold of p(X|H₁)/p(X|H₀)=1/8 and a high threshold of p(X|H₁)/p(X|H₀)=8, where p(X|H₁) is the probability of this result coming from an affected fetus and p(X|H₀) is the null hypothesis of a non-affected fetus.

### Example 4: Clinical Protocol and Validation of Assays

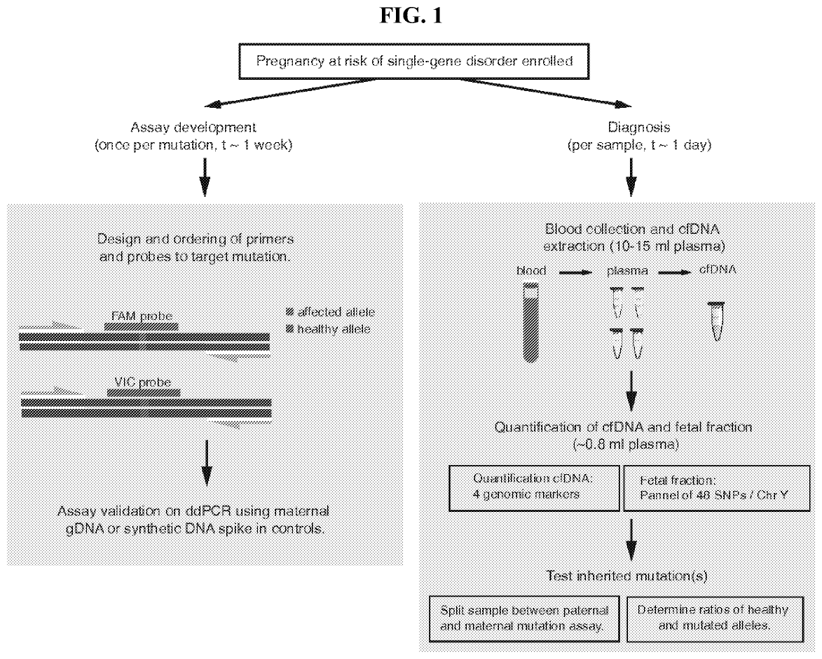

In this study pregnant patients who are carriers of mutations causing autosomal recessive or X-linked disorders were enrolled. The experimental protocol depicted in FIG. 1 was followed to test whether the fetus is affected by the disease. For each pregnancy at risk of a known mutation, primers were designed to amplify the region of the mutation and TaqMan probes labeled with different fluorophores against the healthy and mutated allele at-risk (i.e. single nucleotide mutation, insertion, deletion). The assays were validated using genomic DNA (gDNA) of carriers and non-carriers of the mutation in ddPCR experiments (Example 3 and FIG. 6). Carrier gDNA was obtained from nucleated cells from maternal blood. In order to be able to validate the assays before maternal blood collection, an alternative approach using mixtures of synthetic DNA fragments and spike in experiments (FIG. 2) was used. FIG. 2(a) represents the temperature gradient of 1:1 mixtures of synthetic DNA fragments containing the mutant (FAM) and healthy (VIC) allele for mutation c.835C>T in OTC gene (dbSNP: rs72558455). The optimal temperature for the Taqman assay in ddPCR experiments is highlighted in red. FIG. 2(b) illustrates the spike in controls of the synthetic mutant allele (FAM) in fragmented genomic DNA of a healthy donor. Scatter plots of FAM/VIC fluorescence are shown FIG. 7. (c) Quantification using ddPCR of varying amounts of spike in synthetic DNA (mutant allele) in a background of fragmented gDNA (˜5000 genome equivalents per reaction) from two different healthy donors (red, black). Error bars are obtained from Poisson statistics.

FIG. 6 illustrates the validation of diagnostic assays using fragmented gDNA. (a) Temperature gradient of a Taqman assay targeting mutation c.278-1G>C of F9 gene (Hemophilia B) using fragmented gDNA of an heterozygous carrier of the mutation. Probes targeting the mutant and healthy alleles are labelled with FAM and VIC respectively. The optimal temperature to obtain a good separation between positive and negative droplets in ddPCR experiments is highlighted in red. (b) Scatter plot of FAM/VIC fluorescence for the optimal temperature of the assay selected in (a). Clusters correspond to droplets positive for the mutant allele (blue), the healthy allele (green), both alleles (orange) or none (gray). (c) Scatter plot of FAM/VIC fluorescence for a female control sample. Only droplets positive for the unaffected allele are observed (green cluster). (d) Poisson corrected counts of positive droplets using the diagnostic assay compared to the mean value obtained with the cfDNA quantification assay targeting 4 standard gene marker locations. For a diagnostic assay targeting a single locus in the genome, compatible values in both assays are expected (e.g. two-fold differences from this value might indicate the presence of a pseudogene or copy variants that could interfere with the diagnostic test).

For each incoming sample, cfDNA from ˜30 ml of maternal blood (FIG. 1) was extracted. A quantification assay of the total amount of cfDNA and fetal fraction using Taqman assays targeting 4 genomic markers (cfDNA quantification) was then performed, and a panel of 47 high-variability SNPs and a X/Y chromosome marker (fetal fraction determination). This information was used to decide if a determinative result was possible and to determine the optimal split of sample to test the paternally and maternally-inherited mutations in compound heterozygous conditions, as well as the confidence intervals of the result.

### Example 5: Quantification of cfDNA and Fetal Fraction

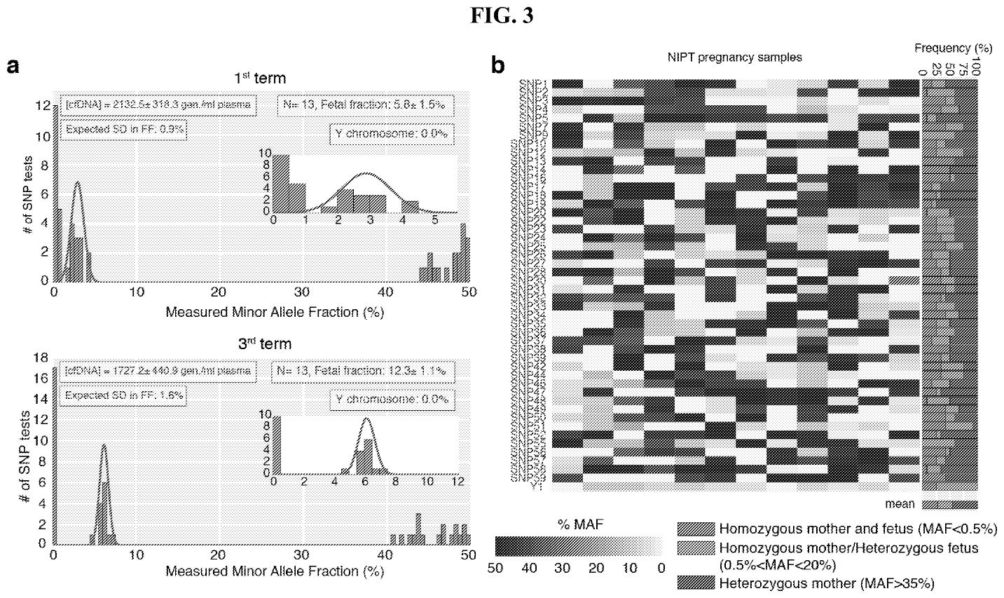

Approximately 7% of each sample to quantify the fetal fraction and total amount of cfDNA was used. For each sample the minor allele fraction (MAF) was used for each SNP in the panel and determined the fetal fraction from the distribution of SNPs that are homozygous for the mother and heterozygous for the fetus, which are found in the range 0.5<MAF<15 (FIG. 3a). This assay allowed for the discrimination of SNPs that are heterozygous for the mother but homozygous for the fetus, which show a characteristic symmetric peak in the range 35<MAF<50 (FIG. 3a). Alternatively, SNPs in FIG. 8 could also be used to improve the estimate if a reduced SNP panel is used. The total quantification of cfDNA was also obtained for each sample, as well as the sex of the fetus (FIG. 3a, insets). FIG. 3(a) illustrates histogram of the MAF for the 47 SNP assays used to determine the fetal fraction. Top (bottom) panel are results from a first (third) term sample of the same pregnancy. The fetal fraction is determined from SNPs that are homozygous for the mother and heterozygous for the fetus (found in the range 0.5%<MAF<20%) and calculated as 2*MAF. A gaussian fit to these SNPs is shown in blue. Inset boxes show the (i) quantification of cfDNA in the sample; (ii) the fetal fraction and number of informative SNP assays (N), (iii) expected error in the fetal fraction, and (iv) sex determination assay. Errors are reported as standard deviation.

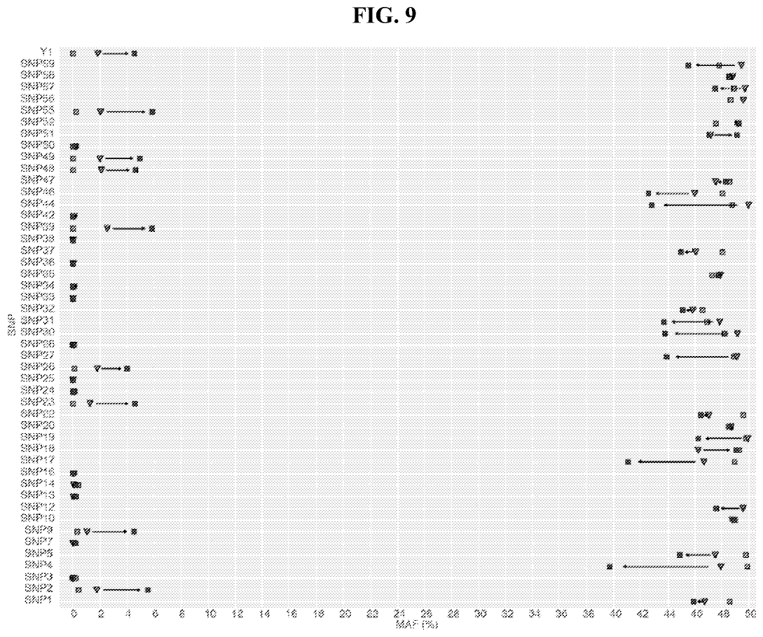

The standard deviation of the fetal fraction measurement was compared to the expected noise due Poisson subsampling (as a limited amount of sample for this measurement was used), finding a good agreement between experimental measurement and theoretical expectation for all samples. The fetal fraction increases with gestational age (FIG. 3a), a result that is also consistently observed for individual SNPs of the panel (FIG. 9). Results for 12 different pregnancies show a distribution of maternal and fetal genotypes suggesting that this panel can be used to determine the fetal fraction in populations of different genetic background (FIG. 3b). FIG. 3 (b) illustrates MAF of the 47-SNP assay for 12 different pregnancies. The right panel shows the frequency of each combination of maternal and fetal genotypes. The recovered distributions are in agreement with the expected results for high-variability SNPs (Heterozygous mother ˜50%, Homozygous mother and fetus ˜25%, Homozygous mother/Heterozygous fetus ˜25%).

FIG. 8 illustrates the Fetal fraction determination using high-variability SNPs. (a) illustrates the probability of a SNP being heterozyogous for the fetus and homozyogus for the mother (i.e. informative SNP for fetal fraction quantification) as a function of its expected MAF in the general population. SNPs selected for the panel lie in the range 0.4<MAF<0.5 (red). (b) Heatmap of the probability of obtaining more than n informative SNPs in a sample (x-axis) as a function of the total number of assays included in the multiplexed SNP panel (y-axis). a total of 47 SNP markers were selected. The shown probability accounts for an additional X/Y chromosome assay with ˜0.5 probability. The number of informative SNPs can be increased by also including SNPs that are heterozygous for the mother but homozygous for the fetus. (c) Distribution of the selected assays for fetal fraction determination (SNPs and X/Y test) across the human genome. FIG. 8 inset represents the size distribution of the human genome per chromosome.

FIG. 9 illustrates the MAF for the fetal fraction assay at different time-points of a pregnancy at risk of MVK deficiency. MAF obtained in the fetal fraction assay for each SNP test for samples collected at: week 17 of pregnancy (blue triangles), week 29 of pregnancy (red squares) and at postpartum (gray squares). Arrows show the variation between the 2^{nd} term and 3^{rd} term sample.

### Example 6: Diagnosis of X-Linked Disorders and Autosomal Recessive Disorders

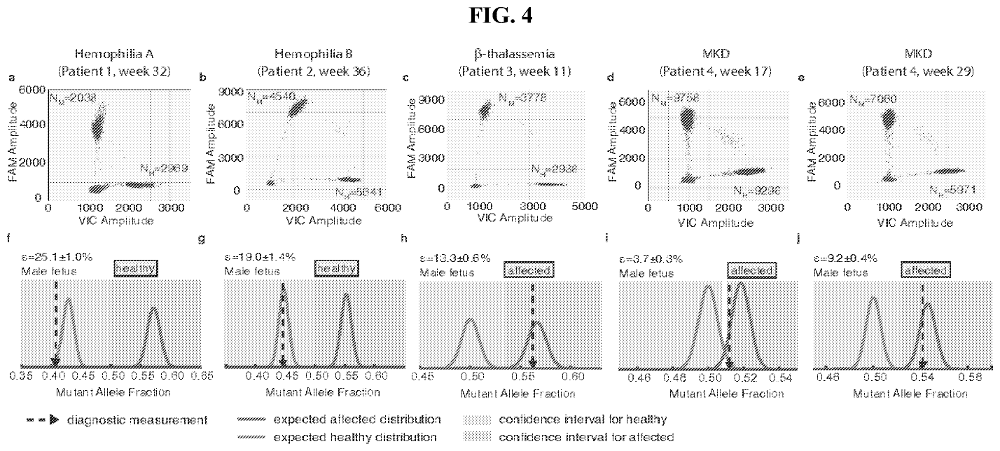

First, the case of X-linked mutations was addressed, where the carrier status of the mother poses a risk for pregnancies carrying a male fetus. Pregnancies at risk of mutations related to hemophilia A, hemophilia B and ornithine transcarbamylase deficiency (OTC) were assessed. Taqman assays targeting these mutations (Table 3) were designed and validated as explained above. The validated assay was then ran for each sample and the Poisson corrected number of mutated (N_{M}) and healthy (N_{H}) alleles in maternal plasma (FIG. 4a-b) was counted. FIG. 4 illustrates the measurement of total counts of mutant (FAM) and healthy (VIC) alleles in maternal plasma using ddPCR for 5 different samples at risk of Hemophilia A (FIG. 4a), Hemophilia B (FIG. 4b), β-thalassemia (FIG. 4c) and mevalonate kinase deficiency (FIG. 4d-e). Clusters correspond to droplets positive for the mutant allele (blue), the healthy allele (green), both alleles (orange) or none (gray). N_{M} and N_{H} are the Poisson corrected counts for the mutant and healthy alleles respectively.

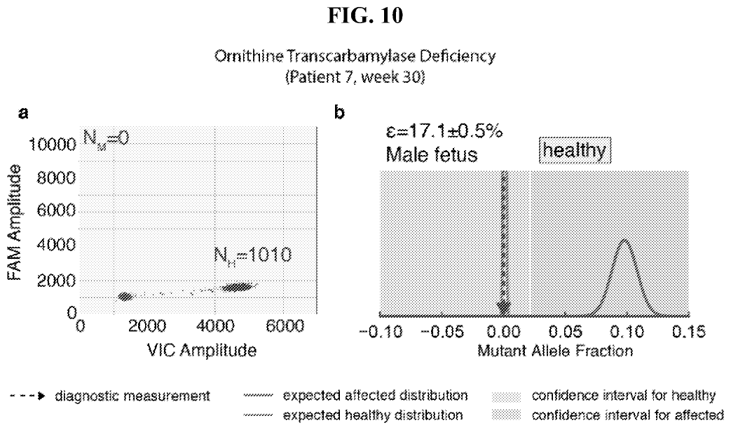

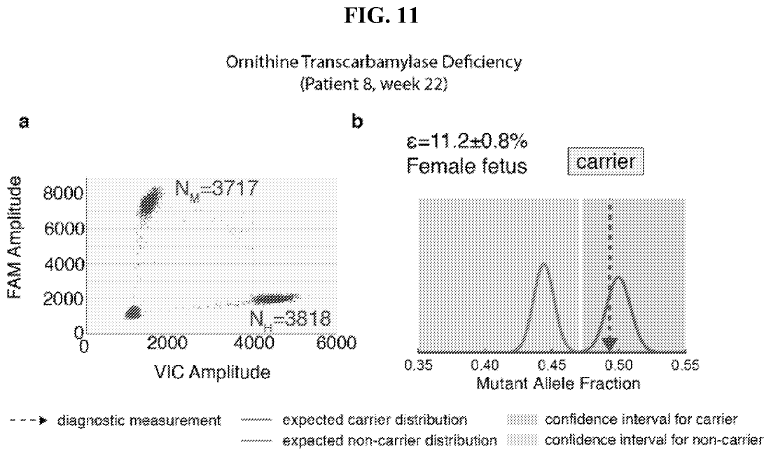

From the measured fetal fraction of each sample, the ratio of mutated and healthy alleles was determined that would be expected for an affected or an unaffected pregnancy as well as its associated error (Example 7). This information was used to compute the expected distributions for an affected or an unaffected pregnancy and compared to the experimentally measured ratio (FIG. 4g-h, blue and green distributions and dotted arrow). The affected or unaffected status of the fetus was determined from the probability of the measurement arising from each distribution using a likelihood ratio classifier (FIG. 4g-h). Using this approach, two pregnancies at-risk of OTC were analyzed. First, a non-carrier mother at-risk due to gamete mosaicism (detected through a previously affected sibling) was tested; it was determined to be an unaffected pregnancy (FIG. 10). Then, a pregnancy carrying a female fetus that was determined to be a carrier of the maternal mutation, and therefore at a partial risk of post-neonatal-onset was analyzed (FIG. 11).

Then the case of autosomal recessive mutations where both mother and father are carriers of the same mutation and therefore at a 25% risk of having an affected pregnancy was addressed. Pregnancies at risk of β-thalassemia and mevalonate kinase deficiency (MKD) were analyzed. To perform the assay, the same approach described for X-linked mutations was followed but the counts and distributions expected for an autosomal recessive disorder was used (Example 7). The assay for each maternal plasma sample was ran, and the number of mutated and healthy alleles was measured (FIG. 4c-d). For both samples the measured ratio was within the confidence intervals for an affected pregnancy (FIG. 4h-i). The affected status of the MKD case was also confirmed in a sample collected later in pregnancy and having a higher fetal fraction (FIG. 4e-j). The dotted arrow corresponds to the measured ratio of mutant allele. The expected distributions for a sample with fetal fraction c and carrying a healthy (affected) fetus is plotted in green (blue) are illustrated. The areas shaded in green and blue correspond to the ratios for which a fetus is determined to be healthy or affected using the ratio classifier. Fetal fraction c is reported as mean±SEM. All measurements were also confirmed in postnatal testing and found to be in agreement with the non-invasive prenatal test.

FIG. 10 illustrates the analysis of a pregnancy at risk of OTC deficiency (c.835C>T) due to maternal gamete mosaicism. FIG. 10(a) illustrates the measurement of total counts of mutant (FAM) and healthy (VIC) alleles in maternal plasma using ddPCR. Only droplets positive for the unaffected allele are observed (green cluster). This is consistent with the fact that the mother is not a carrier of the mutation. NM and NH are the Poisson corrected counts for the mutant and healthy alleles respectively. FIG. 10(b) illustrates the test for the inheritance of the mutation at-risk using a likelihood ratio classifier. As the mother is not a carrier of the mutation, the same approach and statistics explained for a paternally-inherited mutation of an autosomal recessive disorder was used. The dotted arrow corresponds to the measured ratio of mutant allele. The expected distributions for a sample with fetal fraction c and carrying a healthy (affected) fetus are plotted in green (blue). The areas shaded in green and blue correspond to the ratios for which a fetus is determined to be healthy or affected using the ratio classifier.

FIG. 11 illustrates the Analysis of a pregnancy at risk of OTC deficiency (c.67C>T). FIG. 11 (a) illustrates the measurement of total counts of mutant (FAM) and healthy (VIC) alleles in maternal plasma using ddPCR. Clusters correspond to droplets positive for the mutant allele (blue), the healthy allele (green), both alleles (orange) and none (gray). N_{M} and N_{H} are the Poisson corrected counts for the mutant and healthy alleles respectively. FIG. 11(b) illustrates the test for the inheritance of the mutation at-risk using a likelihood ratio classifier. As the fetus is determined to be a female in the fetal fraction assay, a similar approach and statistics as those explained for a maternally-inherited mutation of an autosomal recessive disorder was used. The dotted arrow corresponds to the measured ratio of mutant allele. The expected distributions for a female fetus with fetal fraction c that is a non-carrier (carrier) of the mutation are plotted in green (blue). The areas shaded in green and blue correspond to the ratios for which a fetus is determined to be non-carrier or carrier using the ratio classifier.

### Example 7: Diagnosis of Heterozygous Compound Mutations

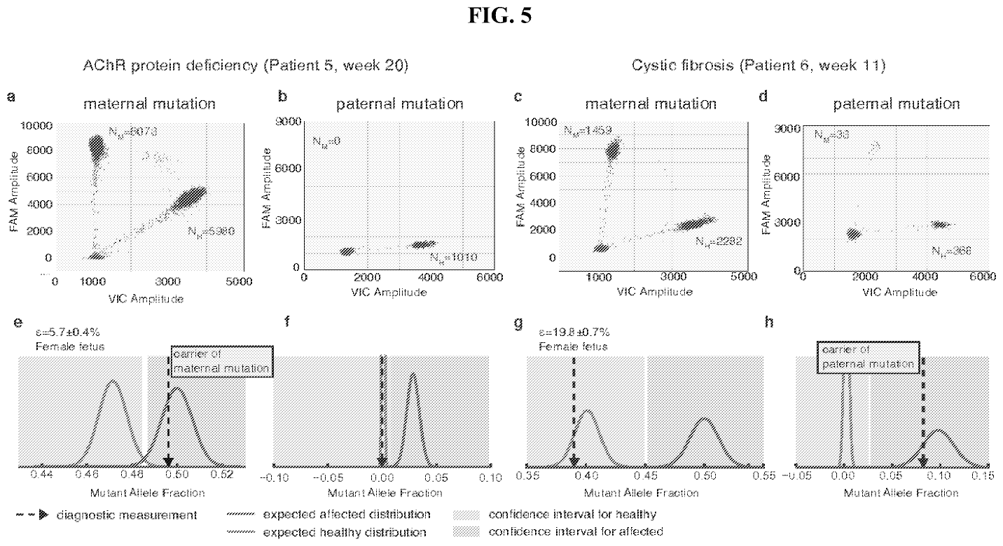

The case of single gene disorders where each parent carries a different mutation affecting the same gene was addressed. Pregnancies at risk of muscle-type acetylcholine receptor (AChR) deficiency (mutations: c459dupA and c753_754delAA), and cystic fibrosis (mutations: ΔF508 and W1282X) were first tested. The latter are the two most common mutations for cystic fibrosis in Ashkenazi Jews, with an estimated combined abundance >75%. For these conditions, the assay for the paternal mutation was ran using enough sample to observe ˜40 counts of the mutated allele in an affected pregnancy. To determine this value, the combined information of the fetal fraction and total cfDNA abundance in maternal plasma was used. From Poisson statistics, this sets the expected result for a fetus that is a carrier of the mutation approximately 6 standard deviations away from a negative result (p<10⁻¹²). The remaining sample was used to detect inheritance of the maternal mutation (Example 7). For each sample, N_{M} and N_{H} for each mutation at-risk was measured (FIG. 5a-d), and determined the genotype of the fetus from the probability of each measurement arising from a carrier or non-carrier using a likelihood ratio classifier (FIG. 5e-h). Measurement of total counts of mutant (FAM) and healthy (VIC) alleles in maternal plasma using ddPCR for a pregnancy at risk of (a,b) AchR deficiency and (c,d) cystic fibrosis. Panels (a) and (c) correspond to the assay testing inheritance of the maternal mutation; panels (b) and (d) correspond to the assay testing inheritance of the paternal mutation. Clusters correspond to droplets positive for the mutant allele (blue), the healthy allele (green), both alleles (orange) or none (gray). N_{M} and N_{H} are the Poisson corrected counts for the mutant and healthy alleles respectively. The dotted arrow corresponds to the measured ratio of mutant allele. The expected distributions for a sample with fetal fraction c and carrying a healthy (affected) fetus is plotted in green (blue). The areas shaded in green and blue correspond to the ratios for which a fetus is determined to be healthy or affected using a ratio classifier. Fetal fraction c is reported as mean±SEM.

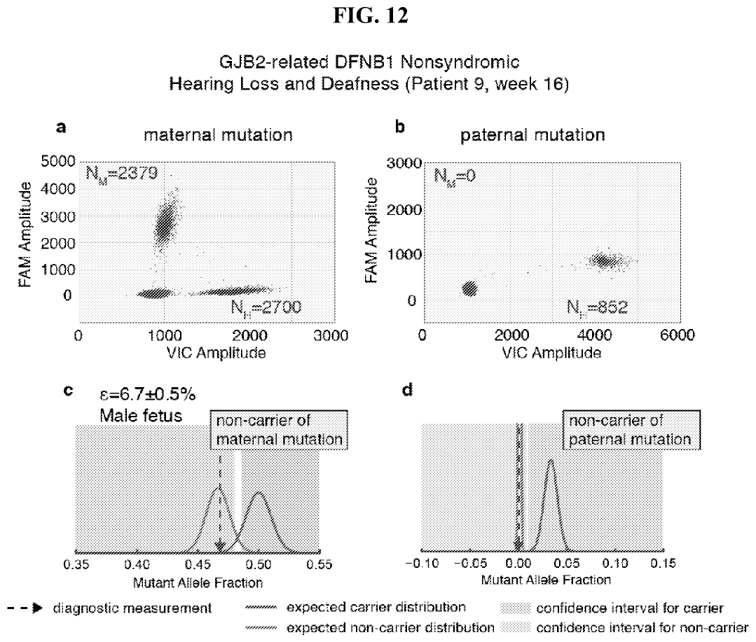

Both pregnancies were determined to have an unaffected fetus, although the fetus at risk of AChR deficiency was determined to be carrier of the maternal mutation whereas the fetus at risk of cystic fibrosis was determined to be carrier of the paternal mutation. Using this approach, a pregnancy at risk of GJB-2 related DFNB1 nonsyndromic hearing loss (mutations: c.71G>A and c.-23+1G>A) at week 16 of gestation (fetal fraction: 6.7±0.5%) was analyzed; it was determined not to be a carrier of the mutated alleles (FIG. 12).

FIG. 12 illustrates the analysis of a pregnancy at risk of GJB2-related DFNB1 nonsyndromic hearing loss due to a heterozygous compound mutation. FIG. 12 (a, b) illustrate the measurement of total counts of mutant (FAM) and healthy (VIC) alleles in maternal plasma using ddPCR. Panels (a) and (b) correspond to the assay testing inheritance of the maternal and paternal mutation respectively. Clusters correspond to droplets positive for the mutant allele (blue), the healthy allele (green), both alleles (orange) or none (gray). N_{M} and N_{H} are the Poisson corrected counts for the mutant and healthy alleles respectively. FIG. 12 (c, d) illustrates the test for the inheritance of the mutation at-risk using a likelihood ratio classifier. The dotted arrow corresponds to the measured ratio of mutant allele. The expected distributions for a sample with fetal fraction c and carrying a healthy (affected) allele for each mutation is plotted in green (blue). The areas shaded in green and blue correspond to the ratios for which a fetus is determined to be a non-carrier or carrier of each mutation using a ratio classifier.

Expected Fractions of Mutated and Wild-Type Alleles from ddPCR Counts and Associated Uncertainities: X-Linked Disease

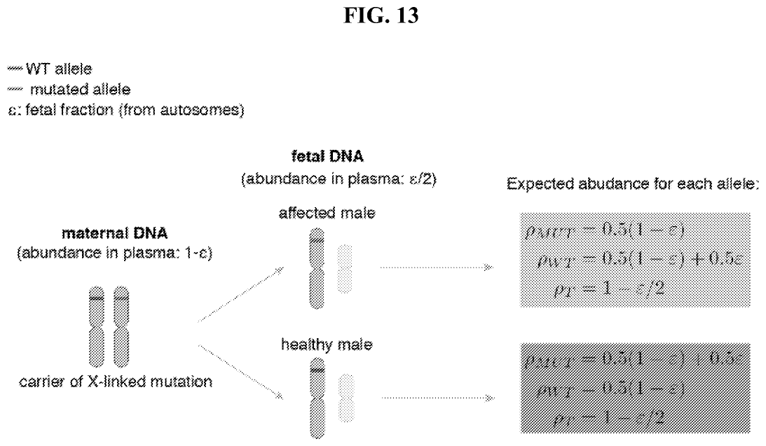

The expected abundances in maternal plasma cfDNA in a pregnancy carrying a male fetus at risk of an X-linked disease can be determined according to the scheme shown in FIG. 13. To estimate the expected fractions of mutated (MUT) and wild-type (WT) alleles, the fraction of fetal DNA must be known from an independent measurement (e.g. panel of SNP assays). Consequently the expected fractions of the mutated (χ_{MUT}) and healthy (χ_{WT}) alleles are presented in FIG. 13.
Affected male fetus:

$${χ}_{MUT} = \frac{{ρ}_{MUT}}{{ρ}_{Tot}} = \frac{1}{2 - ɛ} and {χ}_{WT} = \frac{{ρ}_{WT}}{{ρ}_{Tot}} = \frac{1 - ɛ}{2 - ɛ}$$

Healthy male fetus:

$${χ}_{MUT} = \frac{1 - ɛ}{2 - ɛ} and {χ}_{WT} = \frac{1}{2 - ɛ}$$

Assuming that the number of counts of each allele is an independent Poisson process, for a given ddPCR experiment with a total of NTot counts (NTot=NWT+NMUT), it is expected that:

N_{MUT}=⋅_{MUT}N_{Tot} and N_{WT}=χ_{WT}N_{Tot}

var_{N}_{_{MUT}}=N_{MUT} and var_{N}_{_{WT}}=N_{WT}

The other source of uncertainty on the measurement of fraction of healthy and mutated alleles arises from the error in the measurement of the fetal fraction (δε), which can be measured from the fetal fraction panel assay and taken into account by considering that:

δ_{χWT,ε}=δ_{χMUT,ε}=δε/(2−ε)²

Autosomal Recessive Disease

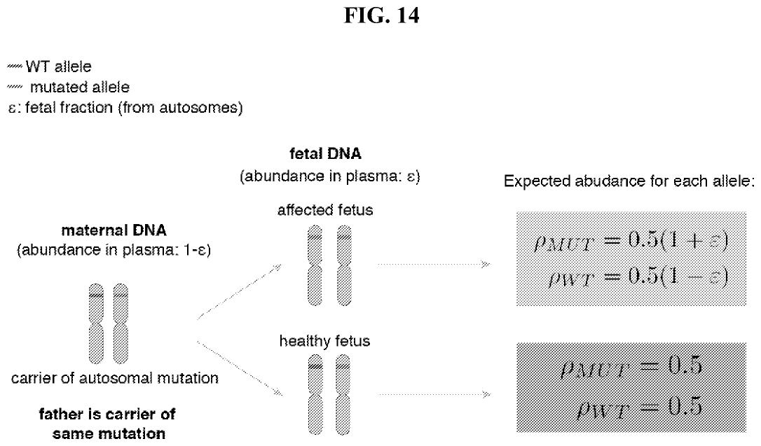

The expected abundances of each allele in maternal plasma cfDNA in a pregnancy at risk of an autosomal recessive disease (male or female) can be determined according to the scheme shown in FIG. 14.

Consequently the expected fractions of the mutated (XMUT) and healthy (WT) alleles are:
Affected fetus:

$${χ}_{MUT} = \frac{{ρ}_{MUT}}{{ρ}_{MUT} + {ρ}_{WT}} = 0.5 ( 1 + ɛ ) and {χ}_{WT} = \frac{{ρ}_{WT}}{{ρ}_{MUT} + {ρ}_{WT}} = 0.5 ( 1 - ɛ )$$

Healthy fetus:

χ_{MUT}=0.5 and χ_{WT}=0.5

Following the same approach described in Example 7, the variances associated to the experimental measurement are

var_{N}_{_{MUT}}=N_{MUT} and var_{N}_{_{WT}}=N_{WT},

and the error arising from the measurement of the fetal fraction is:

δ_{χWT,ε}=δ_{χMUT,ε}=0.5 δε.

### Example 8: Equivalent Blood Draw Required to Achieve Certain False Negative and False Positive Rates

In some embodiments, a sufficient volume of blood collected may be 30 ml with a fetal fraction down to 4%. Table 4 illustrates expected test performance as a function of fetal fraction and blood draw. For instance, in one case, (patient 4, fetal fraction 3.6%, FIG. 4d), the whole sample was used and ˜20,000 counts were obtained, which is in agreement with the blood draw (˜25/30 ml blood) and within a range of expected type I and type II errors of 0.2-1%.

TABLE 4
Equivalent blood draw required to achieve certain false negative and false positive rates.

| | Counts needed (genome eq.) | | | | Equivalent blood draw (ml) | | | |
|---|---|---|---|---|---|---|---|---|
| False positve | 5.0% | 2.0% | 1.0% | 0.2% | 5.0% | 2.0% | 1.0% | 0.2% |
| rate (α) | | | | | | | | |
| False negative | 5.0% | 2.0% | 1.0% | 0.2% | 5.0% | 2.0% | 1.0% | 0.2% |
| rate (β) | | | | | | | | |
| Fetal fraction (ε) | | | | | | | | |
| 3% | 12025 | 18746 | 24053 | 36817 | 18.2 | 28.4 | 36.4 | 55.8 |
| 4% | 6764 | 10545 | 13530 | 20710 | 10.2 | 16.0 | 20.5 | 31.4 |
| 5% | 4329 | 6749 | 8659 | 13254 | 6.6 | 10.2 | 13.1 | 20.1 |
| 6% | 3006 | 4687 | 6013 | 9204 | 4.6 | 7.1 | 9.1 | 13.9 |
| 8% | 1691 | 2636 | 3382 | 5177 | 2.6 | 4.0 | 5.1 | 7.8 |
| 10% | 1082 | 1687 | 2165 | 3314 | 1.6 | 2.6 | 3.3 | 5.0 |

As shown in Table 4, the required number of counts to achieve each false positive and false negative rate may be determined by setting a threshold value between the expected distributions of a positive or negative sample for an autosomal recessive disorder. Equivalent blood draws may be based on the mean concentration of cfDNA found in the samples (1100 counts/ml plasma). To these volumes 1-1.5 mL of blood may be added for fetal fraction and total cfDNA quantification.

In some embodiments, for certain single gene disorders, the sensitivity of the assay could be increased by following high-variability SNPs close to the target mutation (using a similar multiplexing approach as the one used here for the fetal fraction determination). Non limiting examples of such mutations include cystic fibrosis hemophilia, ornithine transcarbamylase deficiency, β-thalassemia, mevalonate kinase deficiency, acetylcholine receptor deficiency and DFNB1 nonsyndromic hearing loss. Alternatively, for many mutations the collection of a moderate volume of blood (2 Streck tubes), may enable a correct classification of samples down to a 5% fetal fraction with false positive and false negative rates ˜0.2%.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of diagnosing a single gene disorder in a fetus comprising:
a) quantifying total cell-free DNA (cfDNA) and a fetal fraction in a non-cellular fraction of a whole blood sample obtained from a pregnant subject, wherein the quantifying comprises an amplification-based multiple single nucleotide polymorphism (SNP) genotyping; and
b) quantifying a ratio of healthy and diseased alleles for a single gene disorder in the non-cellular fraction, wherein the quantifying comprises an amplification-based procedure.

2. The method of claim 1, wherein the pregnant subject is in the first trimester of pregnancy, second trimester of pregnancy or third trimester of pregnancy.

3-7. (canceled)

8. The method of claim 1, wherein the amplification-based SNP genotyping comprises 2 or more SNPs.

9. The method of claim 1, wherein the amplification-based SNP genotyping comprises 14 or more SNPs.

10-11. (canceled)

12. The method of claim 1, wherein a fetal fraction of at least 1.0%, at least 1.5%, at least 2.0%, at least 2.5%, at least 3.0%, at least 3.5% or at least 4.0% is determined in step (a).

13. (canceled)

14. The method of claim 1, further comprising applying a likelihood ratio classifier to the ratio of healthy and diseased alleles to diagnose the single gene disorder in the fetus.

15-18. (canceled)

19. The method of claim 1, wherein the single gene disorder is an X-linked disorder, an autosomal recessive disorder, a compound heterozygous disorder, or a combination thereof.

20-21. (canceled)

22. The method of claim 1, wherein the single gene disorder is a compound heterozygous disorder.

23-49. (canceled)

50. A method of diagnosing a single gene disorder in a fetus comprising:
a) quantifying a fetal fraction in a non-cellular fraction of a whole blood sample obtained from a pregnant subject, wherein the quantifying comprises an amplification-based multiple single nucleotide polymorphism (SNP) genotyping;
b) determining an expected ratio of healthy and diseases alleles for a single gene disorder in the non-cellular fraction;
c) quantifying an actual ratio of healthy and diseased alleles of a single gene disorder in the non-cellular fraction, wherein the quantifying comprises an amplification procedure; and
d) comparing the expected ratio with the actual ratio to diagnose a single gene disorder in a fetus of the pregnant subject.

51. The method of claim 50, wherein the pregnant subject is in the first trimester of pregnancy, second trimester of pregnancy or third trimester of pregnancy.

52-55. (canceled)

56. The method of claim 50, wherein the amplification-based multiple SNP genotyping comprises 2 or more SNPs, 3 or more SNPs, 4 or more SNPs, 5 or more SNPs, 6 or more SNPs, 7 or more SNPs, 8 or more SNPs, 9 or more SNPs, 10 or more SNPs, 11 or more SNPs, 12 or more SNPs, 13 or more SNPs, or 14 or more SNPs.

57-64. (canceled)

65. The method of claim 50, wherein the single gene disorder is an X-linked disorder, an autosomal recessive disorder, a compound heterozygous disorder, or a combination thereof.

66-68. (canceled)

69. The method of claim 50, wherein the single gene disorder is selected from a group consisting of hemophilia A, hemophilia B, ornithine transcarbamylase deficiency (OTC), β-thalassemia, mevalonate kinase deficiency (MKD), muscle-type acetylcholine receptor (AChR) deficiency, cystic fibrosis, and GJB-2 related DFNB1 nonsyndromic hearing loss.

70. The method of claim 50, wherein the whole blood sample is debulked to obtain the non-cellular fraction.

71-90. (canceled)

91. A method of quantifying a fetal fraction in a non-cellular fraction of a whole blood sample from a pregnant subject comprising:
a) performing amplification-based multiple single nucleotide polymorphism (SNP) genotyping and amplification-based chromosomal genotyping of cell-free DNA (cfDNA) in a non-cellular fraction of a whole blood sample from a pregnant subject;
b) quantifying a minor allele fraction (MAF) for each SNP in the SNP genotyping; and
c) determining the fetal fraction as a median of a distribution of SNPs that are: (1) homozygous for the pregnant subject and heterozygous for a fetus of the pregnant subject; and/or (2) heterozygous for the pregnant subject and homozygous for a fetus of the pregnant subject.

92. The method of claim 91, wherein the pregnant subject is in the first trimester of pregnancy, second trimester of pregnancy or third trimester of pregnancy.

93. The method of claim 91, wherein the pregnant subject is in a first trimester of pregnancy.

94-96. (canceled)

97. The method of claim 91, wherein the amplification-based multiple SNP genotyping comprises 2 or more SNPs, 3 or more SNPs, 4 or more SNPs, 5 or more SNPs, 6 or more SNPs, 7 or more SNPs, 8 or more SNPs, 9 or more SNPs, 10 or more SNPs, 11 or more SNPs, 12 or more SNPs, 13 or more SNPs, or 14 or more SNPs.

98-106. (canceled)

107. The method of claim 91, wherein the whole blood sample is debulked to obtain the non-cellular fraction.

108. The method of claim 91, wherein steps (a)-(c) do not require genotyping of the pregnant subject.


SEQUENCE LISTING (52 sequences)

SEQ ID NO: 1 - DNA, 26 bp, Artificial Sequence
  Description of Artificial Sequence Synthetic primer
caagtgctggactcagatgtaactgt

SEQ ID NO: 2 - DNA, 28 bp, Artificial Sequence
  Description of Artificial Sequence Synthetic primer
tgaagtaatgtcagaagctaaaacatca

SEQ ID NO: 3 - DNA, 17 bp, Artificial Sequence
  Description of Artificial Sequence Synthetic probe
tctttaccacactgcac

SEQ ID NO: 4 - DNA, 16 bp, Artificial Sequence
  Description of Artificial Sequence Synthetic probe
tctttagcacattgca

SEQ ID NO: 5 - DNA, 20 bp, Artificial Sequence
  Description of Artificial Sequence Synthetic primer
accactcctgaagtgcactc

SEQ ID NO: 6 - DNA, 21 bp, Artificial Sequence
  Description of Artificial Sequence Synthetic primer
gcgatggttcctcacaagaaa

SEQ ID NO: 7 - DNA, 17 bp, Artificial Sequence
  Description of Artificial Sequence Synthetic probe
attcctcaaaggtcaca

SEQ ID NO: 8 - DNA, 18 bp, Artificial Sequence
  Description of Artificial Sequence Synthetic probe
attcctcgaaggtcacac

SEQ ID NO: 9 - DNA, 23 bp, Artificial Sequence
  Description of Artificial Sequence Synthetic primer
cgtgccaattcaatttcttaacc

SEQ ID NO: 10 - DNA, 25 bp, Artificial Sequence
  Description of Artificial Sequence Synthetic primer
catttaaacatggattggactcaca

SEQ ID NO: 11 - DNA, 17 bp, Artificial Sequence
  Description of Artificial Sequence Synthetic probe
tctcaaacatggagatc

SEQ ID NO: 12 - DNA, 17 bp, Artificial Sequence
  Description of Artificial Sequence Synthetic probe
tctcaaagatggagatc

SEQ ID NO: 13 - DNA, 19 bp, Artificial Sequence
  Description of Artificial Sequence Synthetic primer
tggatgaagttggtggtga

SEQ ID NO: 14 - DNA, 20 bp, Artificial Sequence
  Description of Artificial Sequence Synthetic primer
tggtctccttaaacctgtct

SEQ ID NO: 15 - DNA, 15 bp, Artificial Sequence
  Description of Artificial Sequence Synthetic probe
caggttgctatcaag

SEQ ID NO: 16 - DNA, 15 bp, Artificial Sequence
  Description of Artificial Sequence Synthetic probe
caggttggtatcaag

SEQ ID NO: 17 - DNA, 21 bp, Artificial Sequence
  Description of Artificial Sequence Synthetic primer
tctccatccactcagccacct

SEQ ID NO: 18 - DNA, 20 bp, Artificial Sequence
  Description of Artificial Sequence Synthetic primer
agtgtcgtgggctcctctca

SEQ ID NO: 19 - DNA, 15 bp, Artificial Sequence
  Description of Artificial Sequence Synthetic probe
ctggacagctgagtc

SEQ ID NO: 20 - DNA, 14 bp, Artificial Sequence
  Description of Artificial Sequence Synthetic probe
tggacagccgagtc

SEQ ID NO: 21 - DNA, 18 bp, Artificial Sequence
  Description of Artificial Sequence Synthetic primer
cctgccatcttccgttcc

SEQ ID NO: 22 - DNA, 20 bp, Artificial Sequence
  Description of Artificial Sequence Synthetic primer
gcctcactggaagataaggg

SEQ ID NO: 23 - DNA, 16 bp, Artificial Sequence
  Description of Artificial Sequence Synthetic probe
tctatctcagtcaacc

SEQ ID NO: 24 - DNA, 18 bp, Artificial Sequence
  Description of Artificial Sequence Synthetic probe
tctatctcagtcacctac

SEQ ID NO: 25 - DNA, 20 bp, Artificial Sequence
  Description of Artificial Sequence Synthetic primer
gcaagcccctcttctacgtc

SEQ ID NO: 26 - DNA, 21 bp, Artificial Sequence
  Description of Artificial Sequence Synthetic primer
gatggcgacagaggagatgag

SEQ ID NO: 27 - DNA, 14 bp, Artificial Sequence
  Description of Artificial Sequence Synthetic probe
catcgccccgtgtg

SEQ ID NO: 28 - DNA, 14 bp, Artificial Sequence
  Description of Artificial Sequence Synthetic probe
tcgccccctgtgtg

SEQ ID NO: 29 - DNA, 20 bp, Artificial Sequence
  Description of Artificial Sequence Synthetic primer
tgcctggcaccattaaagaa

SEQ ID NO: 30 - DNA, 20 bp, Artificial Sequence
  Description of Artificial Sequence Synthetic primer
gcatgctttgatgacgcttc

SEQ ID NO: 31 - DNA, 17 bp, Artificial Sequence
  Description of Artificial Sequence Synthetic probe
atatcattggtgtttcc

SEQ ID NO: 32 - DNA, 19 bp, Artificial Sequence
  Description of Artificial Sequence Synthetic probe
atatcatctttggtgtttc

SEQ ID NO: 33 - DNA, 25 bp, Artificial Sequence
  Description of Artificial Sequence Synthetic primer
tgtgtcttgggattcaataactttg

SEQ ID NO: 34 - DNA, 24 bp, Artificial Sequence
  Description of Artificial Sequence Synthetic primer
tttttctggctaagtccttttgct

SEQ ID NO: 35 - DNA, 16 bp, Artificial Sequence
  Description of Artificial Sequence Synthetic probe
aacagtgaaggaaagc

SEQ ID NO: 36 - DNA, 15 bp, Artificial Sequence
  Description of Artificial Sequence Synthetic probe
acagtggaggaaagc

SEQ ID NO: 37 - DNA, 26 bp, Artificial Sequence
  Description of Artificial Sequence Synthetic primer
gcatggaggcaatgtattaattacag

SEQ ID NO: 38 - DNA, 24 bp, Artificial Sequence
  Description of Artificial Sequence Synthetic primer
ggcatcaatttgtaccttcattgt

SEQ ID NO: 39 - DNA, 15 bp, Artificial Sequence
  Description of Artificial Sequence Synthetic probe
aaaaagcggctctag

SEQ ID NO: 40 - DNA, 15 bp, Artificial Sequence
  Description of Artificial Sequence Synthetic probe
aaaaagcggctccag

SEQ ID NO: 41 - DNA, 20 bp, Artificial Sequence
  Description of Artificial Sequence Synthetic primer
tcctgttaaacaatgcagct

SEQ ID NO: 42 - DNA, 20 bp, Artificial Sequence
  Description of Artificial Sequence Synthetic primer
cccaagtctctgaccatcac

SEQ ID NO: 43 - DNA, 17 bp, Artificial Sequence
  Description of Artificial Sequence Synthetic probe
ccgaaaatttcaaacca

SEQ ID NO: 44 - DNA, 17 bp, Artificial Sequence
  Description of Artificial Sequence Synthetic probe
ccgaaaatttcgaacca

SEQ ID NO: 45 - DNA, 20 bp, Artificial Sequence
  Description of Artificial Sequence Synthetic primer
tgaacaaacactccaccagc

SEQ ID NO: 46 - DNA, 20 bp, Artificial Sequence
  Description of Artificial Sequence Synthetic primer
cagccacaacgaggatcata

SEQ ID NO: 47 - DNA, 15 bp, Artificial Sequence
  Description of Artificial Sequence Synthetic probe
cggtgagctagatct

SEQ ID NO: 48 - DNA, 13 bp, Artificial Sequence
  Description of Artificial Sequence Synthetic probe
tgagccagatctt

SEQ ID NO: 49 - DNA, 16 bp, Artificial Sequence
  Description of Artificial Sequence Synthetic primer
caacgccgagaccccc

SEQ ID NO: 50 - DNA, 16 bp, Artificial Sequence
  Description of Artificial Sequence Synthetic primer
gttcctggccgggcag

SEQ ID NO: 51 - DNA, 13 bp, Artificial Sequence
  Description of Artificial Sequence Synthetic probe
acgcagatgagcc

SEQ ID NO: 52 - DNA, 13 bp, Artificial Sequence
  Description of Artificial Sequence Synthetic probe
acgcaggtgagcc